(19) United States
(12) Patent Application Publication
(10) Pub. No.: US 2009/0203574 A1
(43) Pub. Date: Aug. 13, 2009

(54) Method for Modulating Hair Growth

(76) Inventors: Elaine Fuchs, New York, NY (US); Horace Rhee, North Potomac, MD (US)

Correspondence Address: LICATA & TYRRELL P.C., 66 E. MAIN STREET, MARLTON, NJ, 08053, US

(21) Appl. No.: 12/299,628

(22) PCT Filed: May 30, 2007
(86) PCT No.: PCT/US07/69936

§ 371 Date: Nov. 5, 2008

Related U.S. Application Data
(60) Provisional application No. 60/810,007, filed on May 31, 2006.

(51) Int. Cl. A61K 38/00; C12Q 1/02

(52) U.S. Cl. 514/2; 435/29

(57) ABSTRACT

The present invention relates to the use of Lhx2 as a target for modulating the hair growth. Screening assays for identifying agents which increase or decrease the expression or activity of Lhx2 are provided.

### INTRODUCTION

This invention was made in the course of research sponsored by the National Institutes of Health (Grant No. R01-AR050452). The U.S. government may have certain rights in this invention.

### BACKGROUND OF THE INVENTION

Hair follicle morphogenesis involves a temporal series of reciprocal interactions between the ectoderm and its underlying mesenchyme (Hardy (1992) Trends Genet. 8:55; Millar (2002) J. Invest. Dermatol. 118:216; Schmidt-Ullrich and Paus (2005) Bioessays 27:247). During embryogenesis, the skin epidermis develops from a single uniform layer of multipotent cells, separated from the mesenchymally-derived dermis by a basement membrane of extracellular matrix. Cells of this proliferative basal layer can be committed into one of two major lineages. To serve its function as a protective barrier, cells directed towards the epidermal lineage begin a program of terminal differentiation by detaching from the basement membrane, moving outward toward the skin surface, and undergoing metabolic changes to create a keratinized, stratified squamous cell layer. Alternatively, cells of the basal layer can give rise to hair follicles. In response to inductive signals, embryonic hair morphogenesis begins with a localized thickening of epidermal cells and a subsequent bud-like down-growth into the dermis. Known as a hair placode or hair germ, these cells send a reciprocal signal back to the underlying mesenchymal cells to organize into a dermal condensate, the precursor of the dermal papilla. As the hair follicle continues to develop by growing further down into the dermis, a group of rapidly proliferating follicular cells called the matrix surrounds the dermal papilla, forming the hair bulb. Cells losing contact with the hair bulb become the outer root sheath, contiguous with the interfollicular epidermis. The close association between the matrix and dermal papilla within the hair bulb likely results in another set of epithelial-mesenchymal exchange of signals to begin terminal hair differentiation. Specific hair lineages are adopted by the matrix cells as they move upward in concentric rings of cells to form the different layers of the hair follicle, including the inner root sheath and hair shaft. At some point during this morphogenetic process, stem cells residing in the bulge are specified and set aside for the postnatal hair cycle and epidermal repair.

Some of the molecular events involved in hair follicle morphogenesis have been elucidated. In response to an inductive Wnt and an inhibitory Bmp signal (Noggin), small hair placodes bud from the epithelium, giving rise to larger hair germs (DasGupta and Fuchs (1999) Development 126:4557; Huelsken, et al. (2001) Cell 105:533; Botchkarev, et al. (1999) Nature Cell Biol. 1:158; Jamora, et al. (2003) Nature 422:317). In the presence of the mitogen Shh, these hair germs develop further and grow downwards to form a mature follicle that actively produces hair (Chiang, et al. (1999) Dev. Biol. 205:1; Oro and Higgins (2003) Dev. Biol. 255:238; St-Jacques, et al. (1998) Curr. Biol. 8:1058). Although the molecular details of bud formation are still being defined, the general features of this process are repeated at the start of each postnatal hair cycle when multipotent stem cells in the hair follicle bulge become activated to initiate a new round of hair growth. In addition, the early epithelial remodeling to form the hair germ shares many features with the development of other epithelial tissues and organs, including feathers, teeth, and mammary glands (Hogan (1999) Cell 96:225; Pispa and Thesleff (2003) Dev. Biol. 262:195; Yue, et al. (2005) Nature 438:1026). Understanding how tissues form buds which then progress along different lineages is predicated on elucidating the molecular mechanisms that funnel these early signaling pathways into a transcriptional program that drives morphogenesis.

### SUMMARY OF THE INVENTION

The present invention is a method for modulating hair growth by regulating the expression or activity of Lhx2. The present invention also relates to the use of Lhx2 in a screening assay to identify an agent which modulates hair growth. The screening assay involves contacting a test cell expressing a reporter operably linked to an Lhx2 promoter with an agent and detecting expression of the reporter in the test cell, wherein a decrease in reporter expression is indicative of an agent which stimulates hair growth and an increase in reporter expression is indicative of an agent which inhibits hair growth.

### BRIEF DESCRIPTION OF THE DRAWINGS

FIG. 1 shows that Lhx2 maintains follicle stem cells in a quiescent, inactive state. FIG. 1A shows CD34 quantification by flow cytometry in telogen and anagen follicles during the first postnatal cycle. FIG. 1B shows loss of BrdU label retention in KO follicles. Following a 3-day BrdU pulse on d26-28 at the onset of anagen in both wild-type and KO skin grafts, and a 4-week chase when follicles had entered telogen, label retaining cells (LRCs) concentrated in the infrequently dividing bulge stem cells of wild-type follicles, but LRCs were diminished in Lhx2 KO skin. FIG. 1C shows increased BrdU incorporation by KO follicle stem cells. Following a 4-hour BrdU pulse at d40, when wild-type and KO follicles were in mid-anagen of their first postnatal hair cycle, cells were isolated and α6-integrin expressing S-phase cells were quantified by flow cytometry.

FIG. 2 shows the normal program of hair development.

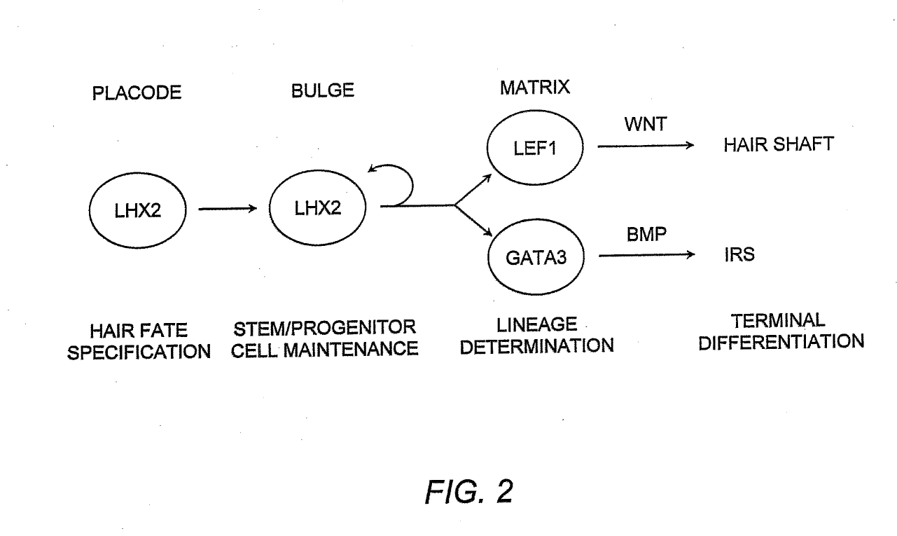

### DETAILED DESCRIPTION OF THE INVENTION

It has now been found that Lhx2 is a transcription factor positioned downstream of signals necessary to specify hair follicle stem cells, but upstream from signals required to drive activated stem cells to terminally differentiate. Using gain and loss of function studies, Lhx2 was found to maintain the growth and undifferentiated properties of hair follicle progenitors. Accordingly, the present invention relates to the use of Lhx2 as a target for modulating hair growth. For example, by increasing the expression or activity of Lhx2, hair follicles can be maintained in a resting or quiescent state thereby preventing or reducing unwanted hair growth, whereas decreasing the expression or activity of Lhx2 can be employed in the stimulation or activation of follicle stem cell proliferation and therefore stimulation of hair growth. Thus, the present invention also embraces screening assays for identifying agents which modulate the expression or activity of Lhx2. Such agents can be identified in in vitro or in vivo screening assays which monitor the activity or expression of Lhx2 (e.g., via reporter protein expression). Agents which can be screened in accordance with the instant assays include the Lhx2 protein or fragments thereof, as well as agonistic or antagonistic anti-Lhx2 antibodies. Ribozymes, siRNA, antisense oligonucleotides and the like can be screened for inhibiting the expression of Lhx2 and small organic molecules can be identified which inhibit or stimulate the expression or activity of Lhx2.

Embryonic hair progenitors were isolated using mice doubly transgenic for a Keratin 14-GFP gene expressed in skin keratinocytes and the Wnt reporter gene TOPGAL, transcribed in hair placodes and germs where β-catenin/Lef1 complexes are active (DasGupta and Fuchs (1999) supra; Vaezi, et al. (2002) Dev. Cell 3:367). In these early hair progenitors, E-cadherin is down-regulated and P-cadherin is upregulated (Jamora, et al. (2003) supra). By embryonic day 17 (E17), dispase was used to separate the epidermis, including hair placodes and germs, from the underlying dermis, which harbored more mature hair pegs and follicles. Using fluorescence-activated cell sorting (FACS) on the epidermal fraction, the early “PCAD+” hair progenitors (K14-GFP+, α6-integrin+, P-cadherin+) were then separated from the “PCAD−” interfollicular epidermis (K14-GFP+, α6-integrin+, P-cadherin−) based on their differential surface P-cadherin expression. Characterization of these two cell populations confirmed their similarities in K5 and β4-integrin expression, but their distinct activities of TOPGAL and expression of known hair placode markers.

The gene expression profiles of purified PCAD+ hair progenitors and PCAD− interfollicular basal keratinocytes were further analyzed using oligonucleotide microarrays. Utilizing fold differences of known hair placode markers as a sensitivity gauge, a 2-fold cut-off was assigned as a genuine difference between the two populations. A total of 1394 probes (660 in PCAD+; 734 in PCAD−) were preferentially expressed greater than 2-fold in one population over the other (Table 1). The Mean Log2 Ratio of Table 1 was calculated for PCAD+ with respect to PCAD− signal values.

A short list of differentially expressed genes relevant to the present study is provided in Table 2. Genes designated with “#” were upregulated and genes designated with “*” were downregulated within the bulge stem cells of postnatal hair follicles compared against the total skin epithelial cell population (Blanpain, et al. (2004) Cell 118:6). A number of these genes have documented roles in either hair morphogenesis (PCAD+) or epidermal differentiation (PCAD−). The interfollicular epidermal population was typified by adhesive and cytoskeletal components, Notch signaling factors, C-myc, Kruppel-like factors, as well as Bmp-responsive transcription factors (Grainyhead-like, Ovol) previously implicated in epidermal differentiation (Fuchs and Raghavan (2002) Nature Rev. Genet. 3:199; Tao, et al. (2005) Development 132:1021; Ting, et al. (2005) Science 308:411; Arnold and Watt (2001) Curr. Biol. 11:558). In contrast, the hair germ signature featured Wnts, Shh, Bmps, Tgfβs, and tyrosine kinase receptor signaling morphogens, as well as a number of different transcription factors. Although some of these transcription factors have not been previously implicated in the specification of skin progenitor fates, others have previously been associated with postnatal genetic hair disorders, including Cutl1, Gli1, Hoxc13, Sox9, Trps1, and Vdr (Millar (2002) supra; Schmidt-Ullrich, et al. (2005) supra).

Unexpectedly, several of the uncharacterized transcription factors on this list were also found to be differentially expressed in the postnatal hair follicle bulge (Blanpain, et al. (2004) supra; Morris, et al. (2004) Nature Biotechnol. 22:411) (Table 2), indicating that the embryonic hair germ may exhibit functional properties similar to adult stem cells. Although the hair germ is committed to a follicular cell fate, it remains undifferentiated like bulge stem cells, yet capable of differentiating into all the lineages of the hair follicle, including the sebaceous gland (Ito, et al. (2005) Nature Med. 11:1351; Levy, et al. Dev. Cell 9:855).

To determine whether the early hair germs may reflect hair follicle stem cells and regulate key steps in progenitor cell differentiation, focus was placed on the transcription factors emanating from the screen that were known to govern developmental cell fate specification in other tissues and organs. Lim-homeodomain transcription factor Lhx2 was of interest since Lhx2 null mutant animals display defects in patterning and cell fate determination during brain development (Porter, et al. (1997) Development 124:2935; Bulchand, et al. (2001) Mech. Dev. 100:165; Hirota and Mombaerts (2004) Proc. Natl. Acad. Sci. USA 101:8751). In addition, they lack definitive erythropoeisis and conversely, hematopoetic progenitor cells can be maintained in vitro by forced expression of Lhx2 (Pinto do, et al. (2002) Blood 99:3939). Lhx2 null animals die between E15.5-E16.5, and a possible role for Lhx2 in skin has not been examined.

Lhx2 was upregulated 18-fold in the PCAD+ population relative to PCAD− population by microarray. Semi-quantitative RT-PCR and in situ hybridization confirmed this marked differential expression. By immunofluorescence, Lhx2 first appeared in early hair placodes, and as morphogenesis progressed, became prominent at the leading front of invaginating hair germs and pegs. As down-growth neared completion and hair differentiation began, Lhx2 concentrated in the upper outer root sheath (ORS) at a presumptive site (bulge) of the developing postnatal follicle stem cell compartment. Concomitantly, expression diminished at the base of the follicle, where highly proliferative matrix cells give rise to the differentiating inner root sheath and hair shaft. In adult follicles, Lhx2 concentrated in the bulge, and as the new hair cycle initiated, Lhx2 extended to the emerging secondary hair germs. Based upon these patterns, Lhx2 appeared to function in specifying the embryonic hair follicle progenitor cells that then persist as bulge stem cells in adult follicles.

To more precisely define Lhx2's role in hair follicle stem cell specification and/or maintenance, its status was examined in various genetic mutant embryos defective in different aspects of hair morphogenesis. In the complete absence of hair follicle induction or bulge maintenance, as reflected in β-catenin conditionally null (cKO) skin, Lhx2 was not expressed. In Shh knockout embryos, where hair germs are specified but unable to progress, Lhx2 expression was dramatically reduced. This positioned Lhx2 downstream of Wnt and Shh, where it could play a role in establishing or expanding the early progenitors necessary for hair follicle morphogenesis.

Bmp signaling is not required for hair follicle induction, even though Bmp ligands and receptors are expressed in embryonic hair germs and in postnatal follicle stem cells. Correspondingly, in BmpR1a cKO skin, Lhx2 was expressed in both embryonic hair germs and the presumptive bulge of developing follicles. Conversely, Bmp signaling is required for differentiation, and in the absence of BmpR1a, proliferating undifferentiated hair progenitor cells accumulate at the follicle base (Andl, et al. (2004) Development 131:2257; Kobielak, et al. (2003) J. Cell Biol. 163:609). Lhx2 was noticeably enhanced in these follicles, with strong staining throughout the ORS and matrix. These cells were also positive for Shh and Lef1. Thus, in the absence of terminal hair differentiation, cells accumulating in postnatal BmpR1a null follicles resembled early embryonic hair follicle progenitors.

If Lhx2 governs the gene expression program of undifferentiated follicle stem cells or their early progenitors, then misexpression of Lhx2 in interfollicular epidermis might result in an induction of hair follicle progenitor genes. Accordingly, K14-Lhx2 transgenic mice were generated to examine this possibility. Although more hair follicles were not induced, Lhx2 markedly suppressed morphological and biochemical signs of epidermal differentiation and failed to produce a functional lipid barrier. Most notable was the induction of Tcf3 and Sox9, two key transcription factors of adult hair follicle stem cells (Merrill, et al. (2001) Genes Dev. 15:1688; Vidal, et al. (2005) Curr. Biol. 15:1340). Lhx2 also suppressed differentiation in tongue epithelium. These findings indicate that Lhx2 can maintain cells in an undifferentiated state, further enforcing the link between Lhx2 and stemness.

If Lhx2 is required for follicle stem cell maintenance, then its absence could alter the ability of hair follicles to form. In support of this notion, E16 Lhx2 null embryos displayed an −40% reduction in overall density of P-cadherin positive hair follicles, with no noticeable defect in the epidermis or embryo size. Marked reduction in follicle density is a feature of other mouse mutants in key hair follicle morphogenetic genes. Although Lhx2 KO follicle density was reduced, Shh, Wnt10b, Bmp2, Bmp4 and Lef1 expression appeared unaffected in those hair placodes and germs that developed. In Lhx2 null skin engraftments, follicles appeared morphologically and biochemically indistinguishable from their wild-type counterparts. Taken together, the gain and loss of function studies indicate that Lhx2, reflecting its expression pattern, functions to specify and maintain hair follicle stem cells, but does not function in their differentiation.

If Lhx2 maintains the undifferentiated state of embryonic and adult follicle stem cells, then Lhx2 null follicles might exhibit alterations in the transition of stem cells from the resting (telogen) to the growing (anagen) phase of the postnatal hair cycle. Using skin grafts, the hair cycles of wild-type and Lhx2 KO follicles were compared. The initial morphogenetic and first postnatal Lhx2 KO hair cycles progressed similarly to wild-type and by 8 weeks, KO follicles had returned to telogen. By contrast, at 11 weeks when most wild-type follicles were still in this extended telogen, KO follicles had precociously entered the next hair cycle. Moreover, upon shaving at 8 weeks, most wild-type hairs remained in telogen while KO hairs consistently and uniformly grew back within 3 weeks, confirming their shortened resting phase.

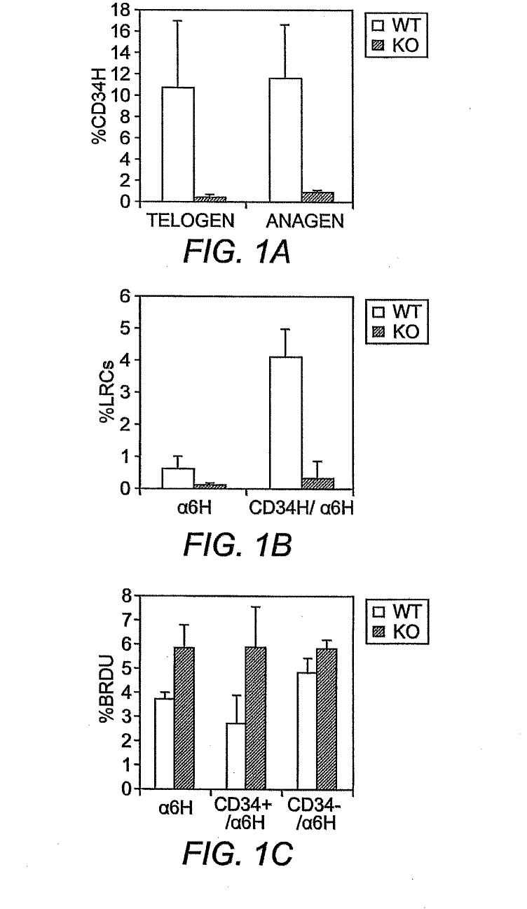

Immunofluorescence and FACS analyses revealed that KO follicles exhibited diminished CD34, a surface marker of bulge stem cells (FIG. 1A) (Blanpain, et al. (2004) supra). This reduction in CD34 was observed irrespective of hair cycle number or stage. Other stem cell markers examined (i.e., Tenascin C and S100A6) were comparably expressed in wild-type and KO bulges.

Although CD34 marks adult stem cells, it is not found in embryonic skin progenitors, suggesting that its reduction could be an indication of enhanced proliferative activity within KO follicle stem cells. This was supported by bromodeoxyuridine (BrdU) pulse-chase experiments conducted prior to marked deviations in hair cycling (FIG. 1B). Only the wild-type follicle bulge compartment retained appreciable BrdU label administered at the onset of anagen and chased for 4 weeks (Blanpain, et al. (2004) supra; Taylor, et al. (2000) Cell 102:451). By contrast, KO hair follicles displayed very few label retaining cells (LRCs), confirmed and quantified by flow cytometry.

The reduction in label retention was accompanied by enhanced proliferation within the KO bulge. After a 4-hour BrdU pulse during full anagen, the percentage of S-phase labeled bulge cells was 2× higher than normal (FIG. 1C). By contrast, the number of S-phase cells in the interfollicular/ORS of wild-type and KO skins was comparable, underscoring the specificity of this hyperproliferation. The elevated proliferative activity of the KO bulge did not appear to alter the overall size of the stem cell niche. It was concluded that without Lhx2, follicle stem cells are more readily activated to proliferate and differentiate along the hair lineage. On the other hand, Lhx2 is not sufficient to induce quiescence as transgenic expression did not suppress proliferation or induce CD34 in the skin epithelium.

By isolating and transcriptionally profiling embryonic hair placodes and interfollicular epidermis, genes implicated in hair development have been identified (Table 1) and novel differences have been uncovered that could be important in orchestrating lineage specification of multipotent skin progenitors. By way of illustration, Lhx2 studies revealed that it functions as a molecular brake in regulating the switch between hair follicle stem cell maintenance and activation. Although follicles can be specified embryonically without Lhx2, their overall numbers are reduced, and Lhx2 null follicles that do form are not proficient in maintaining the resting state and precociously activate. Once committed, cells no longer require or express Lhx2 and progress along a normal program of terminal differentiation.

Finally, Lhx2 is the first identified marker expressed specifically by both embryonic hair placodes and postnatal follicle stem cells of the bulge. Lhx2 now provides a segue to dissect the transcriptional mechanisms that underlie stem cell maintenance within the hair follicle and also serves as a target for modulating hair growth. Further, one or more of the genes identified as being involved in embryonic hair placodes and interfollicular epidermis (Table 1) can be used as a signature of the early hair germ that makes a follicle. Moreover, as with Lhx2, it is contemplated that one of more of the genes listed in Table 1 can be used as targets for modulating hair growth.

The invention is described in greater detail by the following non-limiting examples.

### EXAMPLE 1

### Mice

Lhx2^{-/-}; Shh^{-/-}; BmpR1a^{fl/fl}; Pcad^{-/-}; β-catenin^{fl/fl} are known in the art and were produced according to convention methods. TOPGAL and K14-GFPactin transgenic mice are also known in the art (DasGupta and Fuchs (1999) supra; Vaezi, et al. (2002) Dev. Cell 3:367). Lhx2 transgenic mice were generated by cloning full-length murine Lhx2 cDNA (GENBANK Accession No. NM_010710) into a vector driving expression from the human keratin 14 promoter faithfully expressed in the interfollicular epidermis and outer root sheath of hair follicles (Vasioukhin, et al. (1999) Proc. Natl. Acad. Sci. USA 96:8551).

### EXAMPLE 2

### Engraftment and BrdU Experiments

Full thickness skins were removed from the torsos of sex-matched wild type and Lhx2 null E15.5 embryos, and placed onto the backs of anesthetized female nu/nu (Nude) recipient mice, with each recipient receiving a WT and KO graft. Grafts were secured by sterile gauze and cloth bandages, which were removed after healing (12-13 days). Hairs typically appeared within 1 week after grafting. A total of forty grafts were placed (20 wild-type and 20 KO), and each showed a consistent phenotype dependent on the presence or absence of Lhx2 in the donor skin. 5′-Bromo-2′-deoxyuridine (BrdU; Sigma-Aldrich, St. Louis, Mo.) pulse-chase experiments were performed as described. Intra-peritoneal injections (50 μg/g BrdU) were carried out twice a day for 3 days at day 26 to 28 post-graft and analyzed 4 weeks later for label retention. For cell cycle analysis, mice were injected once with 50 μg/g BrdU at day 40 postgraft and analyzed 4 hours later for BrdU incorporation. A minimum of 4 grafts (two wild-type and two Lhx2 KO) were used for each BrdU experiment. Since K14-Lhx2 transgenic mice died perinatally, newborn back skins were similarly grafted.

### EXAMPLE 3

### Histology, Immunofluorescence, and in situ Hybridizations

Tissues were embedded in OCT and frozen sections were fixed in 4% paraformaldehyde and subjected to immunofluorescence microscopy or hematoxylin/eosin staining. When applicable, the MOM basic kit (Vector laboratories) was used to prevent non-specific binding of mouse monoclonal antibodies (Abs). Antibodies and dilutions used: Lhx2 (rabbit, 1:2500); P- and E-cadherins (rat, 1:100); α6-integrin (rat, 1:100; Pharmingen); K5 (rabbit, 1:500); K1 (rabbit, 1:500); Filaggrin (rabbit, 1:500; Covance); β4-integrin (rat, 1:100; Pharmingen); CD34 (rat, 1:100; Pharmingen); Tenascin C (mouse, 1:500; IBL); S100A6 (rabbit, 1:100; Lab Vision); BrdU (rat, 1:500; Abcam); Tcf3 (guinea pig, 1:300); Sox9 (rabbit, 1:100; Santa Cruz); Lef1 (rabbit, 1:250); Gata3 (mouse, 1:100; Santa Cruz); K6 (rabbit, 1:500); AE13 and AE15 (mouse, 1:50); FITC (1:100; Jackson) or Alexa594 (1:1000; Molecular Probes) conjugated secondary Abs. Nuclei were stained using 4′6′-diamidino-2-phenylindole (DAPI). Imaging was performed using ZEISS AXIOSKOP and AXIOPHOT microscopes equipped with SPOT RT (Diagnostic Instruments) and AXIOCAM (ZEISS) digital cameras, respectively.

### EXAMPLE 4

### Isolation of Hair Progenitors and Flow Cytometry

Back skins from E17.5 K14-GFP embryos were dissected and treated overnight with dispase (Gibco, 0.4 mg/mL) at 4° C., which selectively removed the epidermis, hair placodes, and hair germs from the rest of the skin. This epidermal fraction was treated with 10 mM EDTA, and neutralized cell suspensions were strained (40 μM pores; BD Bioscience). Single cells were resuspended in PBS containing CaCl₂ and MgCl₂ (GIBCO) with 5% FBS, and incubated with primary antibodies coupled to biotin for 30 minutes on ice. After washing with PBS, cells were stained with streptavidin and antibodies directly conjugated to specific fluorophors for 30 minutes on ice. Cells were washed in PBS and resuspended in 300 ng/mL propidium iodide for dead cell exclusion. Cell isolations were performed on FACSVANTAGE SE system equipped with FACS DIVA software (BD Biosciences). Epidermal cells were gated for single events and viability, then sorted according to their expression of K14-GFP, α6-integrin, and P-cadherin. Purity of sorted cells was determined by post-sort FACS analysis and typically exceeded 95%. For grafted skin, single cell suspensions of total skin were isolated by dissecting the graft, mincing into small pieces, and sequential treatment with collagenase (SIGMA) and trypsin (GIBCO) at 37° C. Cells were strained and stained as above. Flow cytometry was performed on FACSORT equipped with CELLQUEST (BD Biosciences). Primary antibodies used for FACS: P-cadherin conjugated to biotin (1:100); α6-integrin coupled to APC or PE (1:100; Pharmingen); β4-integrin (1:100; Pharmingen) coupled to APC; CD34 conjugated to biotin (1:50; Pharmingen). BrdU detection was performed using BrdU Flow Kit (Pharmingen). Cytospin analysis was performed with a Cytospin4 unit (Thermo/Shandon), and stained according to standard methods.

### EXAMPLE 5

### RNA Isolation and Microarray Analyses

Total RNAs from FACS sorted cells were isolated and assessed for quality as described (Rendl, et al. (2005) PLOS Biol. 3:e331). Two rounds of amplification/labeling of 200 ng RNA was performed to obtain biotinylated cRNA for hybridization onto AFFYMETRIX GENECHIP Mouse Genome MOE430 2.0 oligonucleotide microarrays at the Genomics Core Laboratory of Memorial Sloan-Kettering Cancer Center (New York, USA). Two entirely independent samples were used for data analyses. Scanned microarray images were imported into Gene Chip Operating Software (GCOS, AFFYMETRIX) to generate signal values and present/absent calls for each probe set using the MAS 5.0 statistical expression algorithm. Each array was scaled to a target signal of 500 using default analysis parameters. Data files were imported into GENETRAFFIC 3.8 (Iobion Informatics), and replicate microarrays were grouped and compared using the Robust Multi-Chip Analysis algorithm. Genes represented with probe sets ≧2-fold increased in one population over the other and called present in both replicates were considered significant for further analysis.

### EXAMPLE 6

### Semi-Quantitative RT-PCR

Total RNA from FACS sorted cells was isolated as above, quantified with RIBOGREEN (Molecular Probes), and normalized RNA quantities were reverse transcribed with SUPERSCRIPT III using oligo-dT primers (INVITROGEN). PCR amplification of selected genes of interest was performed using primers designed to produce a product spanning exon/intron boundaries.

TABLE 1

| Probe Set ID | UniGene Name | UniGene Symbol | Mean Log2 Ratio |
|---|---|---|---|
| 1425425_a_at | Wnt inhibitory factor 1 | Wif1 | 6.02 |
| 1455280_at | Fras1 related extracellular matrix protein 1 | Frem1 | 5.15 |
| 1416846_a_at | PDZ domain containing RING finger 3 | Pdzrn3 | 4.91 |
| 1427053_at | RIKEN cDNA 5033411B22 gene | 5033411B22Rik | 4.79 |
| 1449530_at | trichorhinophalangeal syndrome I (human) | Trps1 | 4.39 |
| 1427054_s_at | RIKEN cDNA 5033411B22 gene | 5033411B22Rik | 4.36 |
| 1416561_at | glutamic acid decarboxylase 1 | Gad1 | 4.32 |
| 1434286_at | trichorhinophalangeal syndrome I (human) | Trps1 | 4.27 |
| 1418317_at | LIM homeobox protein 2 | Lhx2 | 4.18 |
| 1435349_at | neuropilin 2 | Nrp2 | 4.10 |
| 1446929_at | BTB and CNC homology 2 (Bach2), mRNA | Bach2 | 4.05 |
| 1437667_a_at | BTB and CNC homology 2 | Bach2 | 4.03 |
| 1434195_at | protease, serine, 35 | Prss35 | 3.96 |
| 1457445_at | trichorhinophalangeal syndrome I (human) | Trps1 | 3.90 |
| 1436869_at | sonic hedgehog | Shh | 3.89 |
| 1440304_at | BTB and CNC homology 2 (Bach2), mRNA | Bach2 | 3.80 |
| 1427168_a_at | procollagen, type XIV, alpha 1 | Col14a1 | 3.79 |
| 1450065_at | adenylate cyclase 7 | Adcy7 | 3.79 |
| 1436515_at | RIKEN cDNA E030004N02 gene | E030004N02Rik | 3.74 |
| 1438214_at | trichorhinophalangeal syndrome I (human) | Trps1 | 3.73 |
| 1448870_at | latent transforming growth factor beta binding protein 1 | Ltbp1 | 3.73 |
| 1447343_at | neuropilin 2 | Nrp2 | 3.72 |
| 1420753_at | tolloid-like | Tll1 | 3.70 |
| 1456307_s_at | adenylate cyclase 7 | Adcy7 | 3.69 |
| 1426528_at | neuropilin 2 | Nrp2 | 3.68 |
| 1455961_at | membrane metallo endopeptidase | Mme | 3.64 |
| 1443161_at | Trichorhinophalangeal syndrome I (human) | Trps1 | 3.62 |
| 1442605_at | BTB and CNC homology 2 (Bach2), mRNA | Bach2 | 3.60 |
| 1438532_at | Similar to hemicentin; fibulin 6 (LOC240793), mRNA | NA | 3.59 |
| 1440026_at | Trichorhinophalangeal syndrome I (human) | Trps1 | 3.55 |
| 1440339_at | RIKEN cDNA 4833416E15 gene | 4833416E15Rik | 3.55 |
| 1450922_a_at | transforming growth factor, beta 2 | Tgfb2 | 3.52 |
| 1441657_at | BTB and CNC homology 2 (Bach2), mRNA | Bach2 | 3.51 |
| 1456778_at | neuropilin 2 | Nrp2 | 3.50 |
| 1426640_s_at | tribbles homolog 2 (Drosophila) | Trib2 | 3.49 |
| 1428347_at | cytoplasmic FMR1 interacting protein 2 | Cyfip2 | 3.41 |
| 1450923_at | transforming growth factor, beta 2 | Tgfb2 | 3.41 |
| 1416967_at | SRY-box containing gene 2 | Sox2 | 3.36 |
| 1436002_at | RIKEN cDNA C230013L11 gene | C230013L11Rik | 3.34 |
| 1417155_at | neuroblastoma myc-related oncogene 1 | Nmyc1 | 3.32 |
| 1426766_at | RIKEN cDNA 6330403K07 gene | 6330403K07Rik | 3.30 |
| 1434881_s_at | potassium channel tetramerisation domain containing 12 | Kctd12 | 3.28 |
| 1447174_at | Dachshund 1 (Drosophila) | Dach1 | 3.28 |
| 1455865_at | insulinoma-associated 1 | Insm1 | 3.28 |
| 1435399_at | RIKEN cDNA 2310068J10 gene | 2310068J10Rik | 3.27 |
| 1436555_at | expressed sequence AI158848 | AI158848 | 3.23 |
| 1433743_at | dachshund 1 (Drosophila) | Dach1 | 3.22 |
| 1455277_at | Hedgehog-interacting protein | Hhip | 3.17 |
| 1417336_a_at | synaptotagmin-like 4 | Sytl4 | 3.15 |
| 1423250_a_at | transforming growth factor, beta 2 | Tgfb2 | 3.14 |
| 1454613_at | Dihydropyrimidinase-like 3 | Dpysl3 | 3.11 |
| 1436173_at | deleted in liver cancer 1 | Dlc1 | 3.06 |
| 1440177_at | hypothetical protein 9630027E11 | 9.63E+17 | 3.02 |
| 1425874_at | homeo box C13 | Hoxc13 | 2.95 |
| 1447227_at | Solute carrier family 40 (iron-regulated transporter), member 1 | Slc40a1 | 2.94 |
| 1460602_at | deleted in liver cancer 1 | Dlc1 | 2.94 |
| 1424683_at | RIKEN cDNA 1810015C04 gene | 1810015C04Rik | 2.91 |
| 1450988_at | G protein-coupled receptor 49 | Gpr49 | 2.86 |
| 1437218_at | fibronectin 1 | Fn1 | 2.84 |
| 1435396_at | expressed sequence C85317 | C85317 | 2.81 |
| 1437360_at | Protocadherin 19 | Pcdh19 | 2.81 |
| 1457042_at | Transcribed sequences | NA | 2.81 |
| 1436329_at | early growth response 3 | Egr3 | 2.79 |
| 1456901_at | A disintegrin-like and metalloprotease (reprolysin type) with thro [symbol] | Adamts20 | 2.78 |
| 1429088_at | limb-bud and heart | Lbh | 2.76 |
| 1450770_at | RIKEN cDNA 3632451O06 gene | 3632451O06Rik | 2.72 |
| 1436759_x_at | calponin 3, acidic | Cnn3 | 2.71 |
| 1443263_at | BTB and CNC homology 2 (Bach2), mRNA | Bach2 | 2.69 |
| 1456611_at | RIKEN cDNA D430015B01 gene | D430015B01Rik | 2.69 |
| 1447831_s_at | myotubularin related protein 7 | Mtmr7 | 2.66 |
| 1424704_at | runt related transcription factor 2 | Runx2 | 2.65 |
| 1441956_s_at | Cut-like 1 (Drosophila), transcript variant 2, mRNA (cDNA clone | Cutl1 | 2.65 |
| 1458220_at | deleted in liver cancer 1 | Dlc1 | 2.63 |
| 1423259_at | inhibitor of DNA binding 4 | Id4 | 2.61 |
| 1446141_at | Transforming growth factor, beta 2 | Tgfb2 | 2.60 |
| 1436836_x_at | calponin 3, acidic | Cnn3 | 2.58 |
| 1428572_at | brain abundant, membrane attached signal protein 1 | Basp1 | 2.56 |
| 1425749_at | syntaxin binding protein 6 (amisyn) | Stxbp6 | 2.54 |
| 1443044_at | hypothetical A830091E24 | A830091E24 | 2.54 |
| 1426642_at | fibronectin 1 | Fn1 | 2.53 |
| 1437385_at | collagen and calcium binding EGF domains 1 | Ccbe1 | 2.53 |
| 1419276_at | ectonucleotide pyrophosphatase/phosphodiesterase 1 | Enpp1 | 2.52 |
| 1438303_at | transforming growth factor, beta 2 | Tgfb2 | 2.51 |
| 1458739_at | Latent transforming growth factor beta binding protein 1 | Ltbp1 | 2.51 |
| 1460578_at | FYVE, RhoGEF and PH domain containing 5 | Fgd5 | 2.50 |
| 1436218_at | leucine-rich repeat-containing G protein-coupled receptor 6 | Lgr6 | 2.49 |
| 1454830_at | fibrillin 2 | Fbn2 | 2.48 |
| 1429549_at | procollagen, type XXVII, alpha 1 | Col27a1 | 2.47 |
| 1455570_x_at | calponin 3, acidic | Cnn3 | 2.47 |
| 1429506_at | naked cuticle 1 homolog (Drosophila) | Nkd1 | 2.46 |
| 1450627_at | progressive ankylosis | ank | 2.46 |
| 1420425_at | PR domain containing 1, with ZNF domain | Prdm1 | 2.45 |
| 1419724_at | ectodysplasin-A receptor | Edar | 2.44 |
| 1428209_at | brain expressed X-linked 4 | Bex4 | 2.42 |
| 1428896_at | platelet-derived growth factor receptor-like | Pdgfrl | 2.42 |
| 1433490_s_at | erythrocyte protein band 4.1-like 2 | Epb4.1l2 | 2.42 |
| 1440465_at | Cut-like 1 (Drosophila), transcript variant 2, mRNA (cDNA clone | Cutl1 | 2.42 |
| 1456380_x_at | calponin 3, acidic | Cnn3 | 2.42 |
| 1419485_at | forkhead box C1 | Foxc1 | 2.41 |
| 1456211_at | NACHT, leucine rich repeat and PYD containing 10 | Nalp10 | 2.41 |
| 1456292_a_at | vimentin | Vim | 2.39 |
| 1450928_at | inhibitor of DNA binding 4 | Id4 | 2.38 |
| 1455145_at | 14, 17 days embryo head cDNA, RIKEN full-length enriched libr [symbol] | NA | 2.38 |
| 1423260_at | inhibitor of DNA binding 4 | Id4 | 2.37 |
| 1444780_at | RIKEN cDNA 5330421F07 gene | 5330421F07Rik | 2.37 |
| 1454890_at | angiomotin | Amot | 2.37 |
| 1459546_s_at | ectonucleotide pyrophosphatase/phosphodiesterase 1 | Enpp1 | 2.37 |
| 1434909_at | Ras-related GTP binding D | Rragd | 2.36 |
| 1422869_at | c-mer proto-oncogene tyrosine kinase | Mertk | 2.35 |
| 1455256_at | TRAF2 and NCK interacting kinase | Tnik | 2.34 |
| 1456111_at | hypothetical protein C130036J11 | C130036J11 | 2.33 |
| 1423607_at | lumican | Lum | 2.27 |
| 1434325_x_at | protein kinase, cAMP dependent regulatory, type I beta | Prkar1b | 2.27 |
| 1439852_at | Transcribed sequences | NA | 2.27 |
| 1454742_at | RasGEF domain family, member 1B | Rasgef1b | 2.27 |
| 1433583_at | zinc finger protein 365 | Zfp365 | 2.26 |
| 1416776_at | crystallin, mu | Crym | 2.25 |
| 1417061_at | solute carrier family 40 (iron-regulated transporter), member 1 | Slc40a1 | 2.25 |
| 1443544_at | Transcribed sequences | NA | 2.25 |
| 1416221_at | follistatin-like 1 | Fstl1 | 2.24 |
| 1417359_at | microfibrillar-associated protein 2 | Mfap2 | 2.24 |
| 1438354_x_at | calponin 3, acidic | Cnn3 | 2.23 |
| 1438894_at | Transcribed sequences | NA | 2.23 |
| 1440593_at | 12 days embryo eyeball cDNA, RIKEN full-length enriched libra [symbol] | NA | 2.23 |
| 1434172_at | cannabinoid receptor 1 (brain) | Cnr1 | 2.22 |
| 1439468_at | BTB and CNC homology 2 (Bach2), mRNA | Bach2 | 2.22 |
| 1450505_a_at | RIKEN cDNA 1810015C04 gene | 1810015C04Rik | 2.22 |
| 1457859_at | ADP-ribosylation factor related protein 2 | Arfrp2 | 2.22 |
| 1418028_at | dopachrome tautomerase | Dct | 2.20 |
| 1418723_at | endothelial differentiation, lysophosphatidic acid G-protein-coup [symbol] | Edg7 | 2.20 |
| 1438306_at | ring finger protein 180 | Rnf180 | 2.20 |
| 1448259_at | follistatin-like 1 | Fstl1 | 2.20 |
| 1422912_at | bone morphogenetic protein 4 | Bmp4 | 2.19 |
| 1422643_at | monooxygenase, DBH-like 1 | Moxd1 | 2.18 |
| 1439766_x_at | vascular endothelial growth factor C | Vegfc | 2.17 |
| 1446525_at | Glypican 3, mRNA (cDNA clone MGC: 35964 IMAGE: 4973409) | Gpc3 | 2.17 |
| 1417111_at | mannosidase 1, alpha | Man1a | 2.16 |
| 1438118_x_at | vimentin | Vim | 2.16 |
| 1459619_at | erythrocyte protein band 4.1-like 2 | Epb4.1l2 | 2.16 |
| 1428455_at | procollagen, type XIV, alpha 1 | Col14a1 | 2.15 |
| 1422655_at | patched homolog 2 | Ptch2 | 2.14 |
| 1434136_at | RIKEN cDNA 6332401O19 gene | 6332401O19Rik | 2.14 |
| 1449822_at | atonal homolog 1 (Drosophila) | Atoh1 | 2.14 |
| 1417377_at | immunoglobulin superfamily, member 4A | Igsf4a | 2.12 |
| 1429178_at | odd Oz/ten-m homolog 3 (Drosophila) | Odz3 | 2.12 |
| 1434683_at | Cut-like 1 (Drosophila), transcript variant 2, mRNA (cDNA clone | Cutl1 | 2.12 |
| 1441539_at | 0 day neonate lung cDNA, RIKEN full-length enriched library, cl [symbol] | NA | 2.12 |
| 1443240_at | Glypican 3, mRNA (cDNA clone MGC: 35964 IMAGE: 4973409) | Gpc3 | 2.12 |
| 1460604_at | cytochrome b reductase 1 | Cybrd1 | 2.12 |
| 1437347_at | endothelin receptor type B | Ednrb | 2.11 |
| 1438441_at | Inhibitor of DNA binding 4 | Idb4 | 2.10 |
| 1452309_at | cingulin-like 1 | Cgnl1 | 2.10 |
| 1458205_at | Mannosidase 1, alpha, mRNA (cDNA clone MGC: 18448 IMAGE | Man1a | 2.10 |
| 1422592_at | catenin (cadherin associated protein), delta 2 | Ctnnd2 | 2.09 |
| 1455180_at | expressed sequence AA407270 | AA407270 | 2.09 |
| 1425447_at | dickkopf homolog 4 (Xenopus laevis) | Dkk4 | 2.08 |
| 1427025_at | myotubularin related protein 7 | Mtmr7 | 2.08 |
| 1455262_at | Transcribed sequences | NA | 2.08 |
| 1446088_at | RIKEN cDNA 9430081I23 gene | 9430081I23Rik | 2.07 |
| 1453851_a_at | growth arrest and DNA-damage-inducible 45 gamma | Gadd45g | 2.07 |
| 1423274_at | DEAD/H (Asp-Glu-Ala-Asp/His) box polypeptide 26 | Ddx26 | 2.06 |
| 1423278_at | protein tyrosine phosphatase, receptor type, K | Ptprk | 2.05 |
| 1418176_at | vitamin D receptor | Vdr | 2.04 |
| 1422864_at | runt related transcription factor 1 | Runx1 | 2.04 |
| 1439060_s_at | DNA segment, Chr 11, ERATO Doi 498, expressed | D11Ertd498e | 2.04 |
| 1427086_at | CDNA clone IMAGE: 5028619, partial cds | NA | 2.03 |
| 1433471_at | transcription factor 7, T-cell specific | Tcf7 | 2.03 |
| 1435280_at | expressed sequence AI452195 | AI452195 | 2.03 |
| 1441779_at | RIKEN cDNA 9530006C21 gene | 9530006C21Rik | 2.03 |
| 1429870_at | RIKEN cDNA C630040K21 gene | C630040K21Rik | 2.02 |
| 1455843_at | fucosyltransferase 4 | Fut4 | 2.02 |
| 1459136_at | BTB and CNC homology 2 (Bach2), mRNA | Bach2 | 2.01 |
| 1417411_at | nucleosome assembly protein 1-like 5 | Nap1l5 | 2.00 |
| 1417600_at | solute carrier family 15 (H+/peptide transporter), member 2 | Slc15a2 | 2.00 |
| 1424098_at | ELOVL family member 7, elongation of long chain fatty acids (ye [symbol] | Elovl7 | 2.00 |
| 1435720_at | Potassium voltage-gated channel, Shal-related family, member 3 | Kcnd3 | 2.00 |
| 1438619_x_at | zinc finger, DHHC domain containing 14 | Zdhhc14 | 2.00 |
| 1415949_at | carboxypeptidase E | Cpe | 1.99 |
| 1436076_at | discs, large (Drosophila) homolog-associated protein 1 | Dlgap1 | 1.99 |
| 1444432_at | RIKEN cDNA D330040H18 gene | D330040H18Rik | 1.99 |
| 1446720_at | Activated leukocyte cell adhesion molecule | Alcam | 1.99 |
| 1447567_at | Odd Oz/ten-m homolog 3 (Drosophila) | Odz3 | 1.99 |
| 1423091_a_at | glycoprotein m6b | Gpm6b | 1.98 |
| 1450723_at | ISL1 transcription factor, LIM/homeodomain (islet 1) | Isl1 | 1.98 |
| 1452583_s_at | galactose mutarotase | Galm | 1.98 |
| 1415834_at | dual specificity phosphatase 6 | Dusp6 | 1.97 |
| 1422648_at | solute carrier family 7 (cationic amino acid transporter, y+ syste [symbol] | Slc7a2 | 1.97 |
| 1436551_at | fibroblast growth factor receptor 1 | Fgfr1 | 1.97 |
| 1436791_at | wingless-related MMTV integration site 5A | Wnt5a | 1.97 |
| 1460431_at | glucosaminyl (N-acetyl) transferase 1, core 2 | Gcnt1 | 1.97 |
| 1425779_a_at | T-box 1 | Tbx1 | 1.96 |
| 1440372_at | ADP-ribosylation factor related protein 2 | Arfrp2 | 1.96 |
| 1416632_at | malic enzyme, supernatant | Mod1 | 1.95 |
| 1458249_at | Transcribed sequence with weak similarity to protein ref: NP_08 [symbol] | NA | 1.95 |
| 1424050_s_at | fibroblast growth factor receptor 1 | Fgfr1 | 1.94 |
| 1445711_at | expressed sequence BB163080 | BB163080 | 1.94 |
| 1442740_at | PR domain containing 5 | Prdm5 | 1.93 |
| 1417376_a_at | immunoglobulin superfamily, member 4A | Igsf4a | 1.92 |
| 1417400_at | retinoic acid induced 14 | Rai14 | 1.92 |
| 1446247_at | a disintegrin-like and metalloprotease (reprolysin type) with thro [symbol] | Adamts18 | 1.92 |
| 1448502_at | solute carrier family 16 (monocarboxylic acid transporters), mem [symbol] | Slc16a7 | 1.92 |
| 1458605_at | ADP-ribosylation factor related protein 2 | Arfrp2 | 1.92 |
| 1430264_at | RIKEN cDNA 2610030P05 gene | 2610030P05Rik | 1.91 |
| 1437466_at | activated leukocyte cell adhesion molecule | Alcam | 1.91 |
| 1439554_at | sex comb on midleg homolog 1 | Scmh1 | 1.91 |
| 1446609_at | Transcribed sequences | NA | 1.91 |
| 1442504_at | 0 day neonate thymus cDNA, RIKEN full-length enriched library, | NA | 1.90 |
| 1448765_at | Fyn proto-oncogene | Fyn | 1.90 |
| 1439753_x_at | sine oculis-related homeobox 4 homolog (Drosophila) | Six4 | 1.89 |
| 1426724_at | calponin 3, acidic | Cnn3 | 1.88 |
| 1431118_at | RIKEN cDNA 6720427H10 gene | 6720427H10Rik | 1.88 |
| 1444646_at | RIKEN cDNA 8430420F16 gene | 8430420F16Rik | 1.88 |
| 1449273_at | cytoplasmic FMR1 interacting protein 2 | Cyfip2 | 1.88 |
| 1423584_at | insulin-like growth factor binding protein 7 | Igfbp7 | 1.87 |
| 1437614_x_at | zinc finger, DHHC domain containing 14 | Zdhhc14 | 1.86 |
| 1456621_at | RIKEN cDNA D430015B01 gene | D430015B01Rik | 1.86 |
| 1438861_at | basonuclin 2 | Bnc2 | 1.85 |
| 1441972_at | RIKEN cDNA 6230424C14 gene | 6230424C14Rik | 1.85 |
| 1416688_at | synaptosomal-associated protein 91 | Snap91 | 1.84 |
| 1417378_at | immunoglobulin superfamily, member 4A | Igsf4a | 1.84 |
| 1420965_a_at | ectodermal-neural cortex 1 | Enc1 | 1.84 |
| 1437431_at | Cut-like 1 (Drosophila), transcript variant 2, mRNA (cDNA clone | Cutl1 | 1.84 |
| 1440878_at | runt related transcription factor 1 | Runx1 | 1.84 |
| 1440312_at | ELOVL family member 7, elongation of long chain fatty acids (ye [symbol] | Elovl7 | 1.83 |
| 1440586_at | RIKEN cDNA B430203I24 gene | B430203I24Rik | 1.83 |
| 1448415_a_at | sema domain, immunoglobulin domain (Ig), short basic domain, | Sema3b | 1.83 |
| 1417455_at | transforming growth factor, beta 3 | Tgfb3 | 1.82 |
| 1441206_at | synaptopodin 2 | Synpo2 | 1.81 |
| 1443158_at | Sex comb on midleg homolog 1 | Scmh1 | 1.81 |
| 1443222_at | Thymoma viral proto-oncogene 3 | Akt3 | 1.81 |
| 1449315_at | odd Oz/ten-m homolog 3 (Drosophila) | Odz3 | 1.81 |
| 1455447_at | RIKEN cDNA D430019H16 gene | D430019H16Rik | 1.81 |
| 1428902_at | carbohydrate sulfotransferase 11 | Chst11 | 1.80 |
| 1435832_at | leucine rich repeat containing 4 | Lrrc4 | 1.80 |
| 1428479_at | nuclear factor of activated T-cells, cytoplasmic, calcineurin-depe [symbol] | Nfatc1 | 1.79 |
| 1429359_s_at | RNA binding protein gene with multiple splicing | Rbpms | 1.79 |
| 1436864_at | ADP-ribosylation factor related protein 2 | Arfrp2 | 1.79 |
| 1444992_at | expressed sequence AI120166 | AI120166 | 1.78 |
| 1434073_at | G protein-coupled receptor associated sorting protein 2 | Gprasp2 | 1.77 |
| 1434180_at | pleckstrin homology domain containing, family C (with FERM do [symbol] | Plekhc1 | 1.77 |
| 1436223_at | PREDICTED: integrin beta 8 [Mus musculus], mRNA sequence | Itgb8 | 1.77 |
| 1438896_at | DnaJ (Hsp40) homolog, subfamily C, member 6 | Dnajc6 | 1.77 |
| 1439556_at | neural cell adhesion molecule 1 | Ncam1 | 1.77 |
| 1439895_at | expressed sequence AU021025 | AU021025 | 1.77 |
| 1447040_at | Activated leukocyte cell adhesion molecule | Alcam | 1.77 |
| 1441644_at | Protein tyrosine phosphatase, receptor type, K | Ptprk | 1.76 |
| 1417110_at | mannosidase 1, alpha | Man1a | 1.75 |
| 1420667_at | double C2, beta | Doc2b | 1.75 |
| 1422552_at | reprimo, TP53 dependent G2 arrest mediator candidate | Rprm | 1.75 |
| 1423341_at | chondroitin sulfate proteoglycan 4 | Cspg4 | 1.75 |
| 1436279_at | solute carrier family 26, member 7 | Slc26a7 | 1.75 |
| 1453003_at | sortilin-related receptor, LDLR class A repeats-containing | Sorl1 | 1.75 |
| 1420731_a_at | cysteine and glycine-rich protein 2 | Csrp2 | 1.74 |
| 1440770_at | B-cell leukemia/lymphoma 2 | Bcl2 | 1.74 |
| 1455333_at | tensin 3 | Tns3 | 1.74 |
| 1420589_at | hyaluronan synthase 3 | Has3 | 1.73 |
| 1450191_a_at | SRY-box containing gene 13 | Sox13 | 1.73 |
| 1419486_at | forkhead box C1 | Foxc1 | 1.72 |
| 1421694_a_at | chondroitin sulfate proteoglycan 2 | Cspg2 | 1.72 |
| 1422824_s_at | epidermal growth factor receptor pathway substrate 8 | Eps8 | 1.72 |
| 1456862_at | sine oculis-related homeobox 4 homolog (Drosophila) | Six4 | 1.72 |
| 1417307_at | dystrophin, muscular dystrophy | Dmd | 1.71 |
| 1421598_at | per-hexamer repeat gene 3 | Phxr3 | 1.71 |
| 1429671_at | RIKEN cDNA 2410018M08 gene | 2410018M08Rik | 1.71 |
| 1438473_at | ADP-ribosylation factor related protein 2 | Arfrp2 | 1.71 |
| 1441891_x_at | ELOVL family member 7, elongation of long chain fatty acids (ye [symbol] | Elovl7 | 1.71 |
| 1455535_at | RIKEN cDNA A730017D01 gene | A730017D01Rik | 1.71 |
| 1416165_at | RAB31, member RAS oncogene family | Rab31 | 1.70 |
| 1419700_a_at | prominin 1 | Prom1 | 1.70 |
| 1440840_at | RIKEN cDNA D630004K10 gene | D630004K10Rik | 1.70 |
| 1447204_at | Fibronectin leucine rich transmembrane protein 2 | Flrt2 | 1.70 |
| 1416617_at | acyl-CoA synthetase short-chain family member 1 | Acss1 | 1.69 |
| 1422831_at | fibrillin 2 | Fbn2 | 1.69 |
| 1424097_at | ELOVL family member 7, elongation of long chain fatty acids (ye [symbol] | Elovl7 | 1.69 |
| 1441573_at | sex comb on midleg homolog 1 | Scmh1 | 1.69 |
| 1442467_at | RIKEN cDNA 5330421F07 gene | 5330421F07Rik | 1.69 |
| 1417585_at | nucleoporin 210 | Nup210 | 1.68 |
| 1418847_at | arginase type II | Arg2 | 1.68 |
| 1423858_a_at | 3-hydroxy-3-methylglutaryl-Coenzyme A synthase 2 | Hmgcs2 | 1.68 |
| 1434557_at | huntingtin interacting protein 1 | Hip1 | 1.68 |
| 1438975_x_at | zinc finger, DHHC domain containing 14 | Zdhhc14 | 1.68 |
| 1449033_at | tumor necrosis factor receptor superfamily, member 11b (osteop [symbol] | Tnfrsf11b | 1.68 |
| 1433699_at | tumor necrosis factor, alpha-induced protein 3 | Tnfaip3 | 1.67 |
| 1435941_at | rhomboid, veinlet-like 4 (Drosophila) | Rhbdl4 | 1.67 |
| 1439191_at | follistatin-like 1 | Fstl1 | 1.67 |
| 1441091_at | Transcribed sequences | NA | 1.67 |
| 1458324_x_at | 16 days embryo head cDNA, RIKEN full-length enriched library, | NA | 1.67 |
| 1423407_a_at | fibulin 2 | Fbln2 | 1.66 |
| 1433642_at | ADP-ribosylation factor related protein 2 | Arfrp2 | 1.66 |
| 1454799_at | Transcribed sequences | NA | 1.65 |
| 1420377_at | sialyltransferase 8 (alpha-2,8-sialyltransferase) B | Siat8b | 1.64 |
| 1427256_at | chondroitin sulfate proteoglycan 2 | Cspg2 | 1.64 |
| 1437967_at | Titin | Ttn | 1.64 |
| 1446196_at | high mobility group AT-hook 2 | Hmga2 | 1.64 |
| 1452366_at | RIKEN cDNA 4732435N03 gene | 4732435N03Rik | 1.64 |
| 1453514_at | glycoprotein m6b | Gpm6b | 1.64 |
| 1418210_at | profilin 2 | Pfn2 | 1.63 |
| 1443086_at | activated leukocyte cell adhesion molecule | Alcam | 1.63 |
| 1422823_at | epidermal growth factor receptor pathway substrate 8 | Eps8 | 1.62 |
| 1427257_at | chondroitin sulfate proteoglycan 2 | Cspg2 | 1.62 |
| 1430307_a_at | malic enzyme, supernatant | Mod1 | 1.62 |
| 1438841_s_at | arginase type II | Arg2 | 1.62 |
| 1440354_at | ELOVL family member 7, elongation of long chain fatty acids (ye [symbol] | Elovl7 | 1.62 |
| 1440678_at | Latent transforming growth factor beta binding protein 1 | Ltbp1 | 1.62 |
| 1442680_at | Neural cell adhesion molecule 1 | Ncam1 | 1.62 |
| 1453041_at | Trp53 inducible protein 5 | Trp53i5 | 1.62 |
| 1423277_at | protein tyrosine phosphatase, receptor type, K | Ptprk | 1.61 |
| 1443837_x_at | B-cell leukemia/lymphoma 2 | Bcl2 | 1.61 |
| 1448566_at | solute carrier family 40 (iron-regulated transporter), member 1 | Slc40a1 | 1.61 |
| 1426865_a_at | neural cell adhesion molecule 1 | Ncam1 | 1.60 |
| 1429893_at | RIKEN cDNA 2810004A10 gene | 2810004A10Rik | 1.60 |
| 1433776_at | lipoma HMGIC fusion partner | Lhfp | 1.60 |
| 1440758_at | 7 days neonate cerebellum cDNA, RIKEN full-length enriched lib [symbol] | NA | 1.60 |
| 1443394_at | RIKEN cDNA 9230105E05 gene | 9230105E05Rik | 1.60 |
| 1447491_at | Leucine rich repeat containing 5 | Lrrc5 | 1.60 |
| 1457275_at | desmuslin | Dmn | 1.60 |
| 1416041_at | serum/glucocorticoid regulated kinase | Sgk | 1.59 |
| 1417505_s_at | interleukin 11 receptor, alpha chain 1 | Il11ra1 | 1.59 |
| 1443018_at | Neural cell adhesion molecule 1 | Ncam1 | 1.59 |
| 1450061_at | ectodermal-neural cortex 1 | Enc1 | 1.59 |
| 1453102_at | fibronectin leucine rich transmembrane protein 3 | Flrt3 | 1.59 |
| 1453191_at | procollagen, type XXVII, alpha 1 | Col27a1 | 1.59 |
| 1419044_at | contactin associated protein 4 | Cntnap4 | 1.58 |
| 1426301_at | activated leukocyte cell adhesion molecule | Alcam | 1.58 |
| 1427182_s_at | DNA segment, Chr 18, ERATO Doi 653, expressed | D18Ertd653e | 1.58 |
| 1442174_at | tetraspanin 18 | Tspan18 | 1.58 |
| 1417520_at | nuclear factor, erythroid derived 2, like 3 | Nfe2l3 | 1.57 |
| 1417621_at | nuclear factor of activated T-cells, cytoplasmic, calcineurin-depe [symbol] | Nfatc1 | 1.57 |
| 1425761_a_at | nuclear factor of activated T-cells, cytoplasmic, calcineurin-depe [symbol] | Nfatc1 | 1.57 |
| 1437760_at | UDP-N-acetyl-alpha-D-galactosamine:polypeptide N-acetylgalac [symbol] | Galnt12 | 1.57 |
| 1450781_at | high mobility group AT-hook 2 | Hmga2 | 1.57 |
| 1452287_at | Musashi homolog 1(Drosophila) | Msi1h | 1.57 |
| 1421818_at | B-cell leukemia/lymphoma 6 | Bcl6 | 1.56 |
| 1441769_at | ADP-ribosylation factor related protein 2 | Arfrp2 | 1.56 |
| 1448788_at | antigen identified by monoclonal antibody MRC OX-2 | Mox2 | 1.56 |
| 1457687_at | B-cell leukemia/lymphoma 2 | Bcl2 | 1.56 |
| 1420928_at | beta galactoside alpha 2,6 sialyltransferase 1 | St6gal1 | 1.55 |
| 1436501_at | mitochondrial tumor suppressor 1 | Mtus1 | 1.55 |
| 1445211_at | Sex comb on midleg homolog 1 | Scmh1 | 1.55 |
| 1419468_at | C-type lectin domain family 14, member a | Clec14a | 1.54 |
| 1448877_at | distal-less homeobox 2 | Dlx2 | 1.54 |
| 1451359_at | cDNA sequence BC005662 | BC005662 | 1.54 |
| 1458113_at | RIKEN cDNA 9530019H20 gene | 9530019H20Rik | 1.54 |
| 1418175_at | vitamin D receptor | Vdr | 1.53 |
| 1424114_s_at | laminin B1 subunit 1 | Lamb1-1 | 1.53 |
| 1433596_at | DnaJ (Hsp40) homolog, subfamily C, member 6 | Dnajc6 | 1.53 |
| 1451435_at | cut-like 1 (Drosophila) | Cutl1 | 1.53 |
| 1456404_at | a disintegrin-like and metalloprotease (reprolysin type) with thro [symbol] | Adamts5 | 1.53 |
| 1457434_s_at | protein tyrosine phosphatase-like (proline instead of catalytic arg [symbol] | Ptpla | 1.52 |
| 1417319_at | poliovirus receptor-related 3 | Pvrl3 | 1.51 |
| 1417780_at | longevity assurance homolog 4 (S. cerevisiae) | Lass4 | 1.51 |
| 1426864_a_at | neural cell adhesion molecule 1 | Ncam1 | 1.51 |
| 1436959_x_at | nasal embryonic LHRH factor | Nelf | 1.51 |
| 1444993_at | Ring finger protein 32 | Rnf32 | 1.51 |
| 1460595_at | 16 days embryo head cDNA, RIKEN full-length enriched library, | NA | 1.51 |
| 1417673_at | growth factor receptor bound protein 14 | Grb14 | 1.50 |
| 1424113_at | laminin B1 subunit 1 | Lamb1-1 | 1.50 |
| 1442625_at | Transcribed sequence with weak similarity to protein ref: NP_08 [symbol] | NA | 1.50 |
| 1424890_at | basonuclin 1 | Bnc1 | 1.49 |
| 1426300_at | activated leukocyte cell adhesion molecule | Alcam | 1.49 |
| 1436512_at | ADP-ribosylation factor-like 7 | Arl7 | 1.49 |
| 1441391_at | Guanine nucleotide binding protein (G protein), beta polypeptide | Gnb1l | 1.49 |
| 1455096_at | fibronectin leucine rich transmembrane protein 2 | Flrt2 | 1.49 |
| 1428387_at | acyl-CoA synthetase long-chain family member 3 | Acsl3 | 1.48 |
| 1430320_at | dystrophin, muscular dystrophy | Dmd | 1.48 |
| 1448323_a_at | biglycan | Bgn | 1.48 |
| 1450990_at | glypican 3 | Gpc3 | 1.48 |
| 1460070_at | Carbohydrate sulfotransferase 2 | Chst2 | 1.48 |
| 1423331_a_at | poliovirus receptor-related 3 | Pvrl3 | 1.47 |
| 1425007_at | zinc finger protein 566 | Zfp566 | 1.47 |
| 1439341_at | Transcribed sequences | NA | 1.47 |
| 1441509_at | RIKEN cDNA A130009I22 gene | A130009I22Rik | 1.47 |
| 1447547_at | latent transforming growth factor beta binding protein 1 | Ltbp1 | 1.47 |
| 1459420_at | Transcribed sequences | NA | 1.47 |
| 1418835_at | pleckstrin homology-like domain, family A, member 1 | Phlda1 | 1.46 |
| 1429310_at | fibronectin leucine rich transmembrane protein 3 | Flrt3 | 1.46 |
| 1433626_at | phospholipid scramblase 4 | Plscr4 | 1.46 |
| 1434848_at | G protein-coupled receptor 27 | Gpr27 | 1.46 |
| 1436419_a_at | RIKEN cDNA 1700097N02 gene | 1700097N02Rik | 1.46 |
| 1440555_at | Ras-related GTP binding D | Rragd | 1.46 |
| 1448673_at | poliovirus receptor-related 3 | Pvrl3 | 1.46 |
| 1449286_at | netrin G1 | Ntng1 | 1.46 |
| 1450044_at | frizzled homolog 7 (Drosophila) | Fzd7 | 1.46 |
| 1416405_at | biglycan | Bgn | 1.45 |
| 1436917_s_at | G-protein signalling modulator 1 (AGS3-like, C. elegans) | Gpsm1 | 1.45 |
| 1440506_at | Solute carrier family 7 (cationic amino acid transporter, y+ syste [symbol] | Slc7a2 | 1.45 |
| 1451835_at | SRY-box containing gene 21 | Sox21 | 1.45 |
| 1419470_at | guanine nucleotide binding protein, beta 4 | Gnb4 | 1.44 |
| 1422708_at | phosphoinositide-3-kinase, catalytic, gamma polypeptide | Pik3cg | 1.44 |
| 1439793_at | Gap junction membrane channel protein alpha 3 (Gja3), mRNA | Gja3 | 1.44 |
| 1443870_at | ATP-binding cassette, sub-family C (CFTR/MRP), member 4 | Abcc4 | 1.44 |
| 1446380_at | RIKEN cDNA 9430076C15 gene | 9430076C15Rik | 1.44 |
| 1447295_at | Cut-like 1 (Drosophila), transcript variant 2, mRNA (cDNA clone | Cutl1 | 1.44 |
| 1450064_at | formin 2 | Fmn2 | 1.44 |
| 1455995_at | DNA segment, Chr 10, Brigham & Women's Genetics 1379 expr [symbol] | D10Bwg1379e | 1.44 |
| 1456811_at | CXXC finger 4 | Cxxc4 | 1.44 |
| 1420872_at | guanylate cyclase 1, soluble, beta 3 | Gucy1b3 | 1.43 |
| 1430876_at | RIKEN cDNA 2610312I10 gene | 2610312I10Rik | 1.43 |
| 1443194_at | RIKEN cDNA 5930430O07 gene | 5930430O07Rik | 1.43 |
| 1453545_at | RIKEN cDNA 5730492I20 gene | 5730492I20Rik | 1.43 |
| 1426258_at | sortilin-related receptor, LDLR class A repeats-containing | Sorl1 | 1.42 |
| 1435701_at | 10 days neonate medulla oblongata cDNA, RIKEN full-length en [symbol] | NA | 1.42 |
| 1442411_at | Glucocorticoid induced transcript 1 | Glcci1 | 1.42 |
| 1454824_s_at | mitochondrial tumor suppressor 1 | Mtus1 | 1.42 |
| 1437122_at | B-cell leukemia/lymphoma 2 | Bcl2 | 1.41 |
| 1437422_at | sema domain, seven thrombospondin repeats (type 1 and type [symbol] | Sema5a | 1.41 |
| 1422851_at | high mobility group AT-hook 2 | Hmga2 | 1.40 |
| 1437492_at | RIKEN cDNA 9430023B20 gene | 9430023B20Rik | 1.40 |
| 1442992_at | hypothetical LOC403343 | 130004C03 | 1.40 |
| 1444763_at | protein tyrosine phosphatase, receptor type, K | Ptprk | 1.40 |
| 1446583_at | NA | NA | 1.40 |
| 1448557_at | RIKEN cDNA 1200015N20 gene | 1200015N20Rik | 1.40 |
| 1454866_s_at | chloride intracellular channel 6 | Clic6 | 1.40 |
| 1434776_at | sema domain, seven thrombospondin repeats (type 1 and type [symbol] | Sema5a | 1.39 |
| 1435207_at | DIX domain containing 1 | Dixdc1 | 1.39 |
| 1437403_at | RIKEN cDNA E130306M17 gene | E130306M17Rik | 1.39 |
| 1443827_x_at | cDNA sequence BC004044 | BC004044 | 1.39 |
| 1416892_s_at | RIKEN cDNA 3110001A13 gene | 3110001A13Rik | 1.38 |
| 1438989_s_at | RIKEN cDNA B130021B11 gene | B130021B11Rik | 1.38 |
| 1456315_a_at | protein tyrosine phosphatase-like (proline instead of catalytic arg [symbol] | Ptpla | 1.38 |
| 1418608_at | calmodulin-like 3 | Calml3 | 1.37 |
| 1426858_at | inhibin beta-B | Inhbb | 1.37 |
| 1433779_at | cancer susceptibility candidate 4 | Casc4 | 1.37 |
| 1445697_at | Similar to development- and differentiation-enhancing factor 2; F [symbol] | — | 1.37 |
| 1451899_a_at | general transcription factor II I repeat domain-containing 1 | Gtf2ird1 | 1.37 |
| 1456095_at | Tyrosinase (Tyr), mRNA | Tyr | 1.37 |
| 1427579_at | rhomboid, veinlet-like 4 (Drosophila) | Rhbdl4 | 1.36 |
| 1429987_at | RIKEN cDNA 9930013L23 gene | 9930013L23Rik | 1.36 |
| 1435892_at | RIKEN cDNA D430002O22 gene | D430002O22Rik | 1.36 |
| 1437574_at | a disintegrin-like and metalloprotease (reprolysin type) with thror [symbol] | Adamts18 | 1.36 |
| 1441947_x_at | cDNA sequence BC033915 | BC033915 | 1.36 |
| 1446772_at | Transcribed sequences | NA | 1.36 |
| 1423110_at | procollagen, type I, alpha 2 | Col1a2 | 1.35 |
| 1439795_at | G protein-coupled receptor 64 | Gpr64 | 1.35 |
| 1448665_at | dystrophin, muscular dystrophy | Dmd | 1.35 |
| 1448698_at | cyclin D1 | Ccnd1 | 1.35 |
| 1460364_at | general transcription factor II I repeat domain-containing 1 | Gtf2ird1 | 1.35 |
| 1448669_at | dickkopf homolog 3 (Xenopus laevis) | Dkk3 | 1.34 |
| 1417937_at | dapper homolog 1, antagonist of beta-catenin (xenopus) | Dact1 | 1.33 |
| 1426008_a_at | solute carrier family 7 (cationic amino acid transporter, y+ syste [symbol] | Slc7a2 | 1.33 |
| 1456515_s_at | transcription factor-like 5 (basic helix-loop-helix) | Tcfl5 | 1.33 |
| 1436993_x_at | profilin 2 | Pfn2 | 1.32 |
| 1438702_at | fibronectin leucine rich transmembrane protein 2 | Flrt2 | 1.32 |
| 1452106_at | nephronectin | Npnt | 1.32 |
| 1455436_at | DIRAS family, GTP-binding RAS-like 2 | Diras2 | 1.32 |
| 1417782_at | longevity assurance homolog 4 (S. cerevisiae) | Lass4 | 1.31 |
| 1423635_at | bone morphogenetic protein 2 | Bmp2 | 1.31 |
| 1429001_at | pirin | Pir | 1.31 |
| 1444306_at | Musashi homolog 1(Drosophila) | Msi1h | 1.31 |
| 1448901_at | carboxypeptidase X 1 (M14 family) | Cpxm1 | 1.31 |
| 1429053_at | RIKEN cDNA 1110012J17 gene | 1110012J17Rik | 1.30 |
| 1429285_at | serine (or cysteine) proteinase inhibitor, clade A (alpha-1 antipro | Serpina9 | 1.30 |
| 1434709_at | RIKEN cDNA C130076O07 gene | C130076O07Rik | 1.30 |
| 1436181_at | development and differentiation enhancing factor 2 | Ddef2 | 1.30 |
| 1453595_at | RIKEN cDNA 2900064B18 gene | 2900064B18Rik | 1.30 |
| 1454788_at | ADP-ribosylation factor-like 7 | Arl7 | 1.30 |
| 1455325_at | RIKEN cDNA A230057G18 gene | A230057G18Rik | 1.30 |
| 1458947_at | Fanconi anemia, complementation group C | Fancc | 1.30 |
| 1424625_a_at | DENN/MADD domain containing 1A | Dennd1a | 1.29 |
| 1432103_a_at | SH3-domain GRB2-like 3 | Sh3gl3 | 1.29 |
| 1434628_a_at | rhophilin, Rho GTPase binding protein 2 | Rhpn2 | 1.29 |
| 1436761_s_at | RIKEN cDNA 1200015N20 gene | 1200015N20Rik | 1.29 |
| 1437889_x_at | biglycan | Bgn | 1.29 |
| 1431202_at | hect domain and RLD 3 | Herc3 | 1.28 |
| 1416842_at | glutathione S-transferase, mu 5 | Gstm5 | 1.27 |
| 1423885_at | laminin, gamma 1 | Lamc1 | 1.27 |
| 1429514_at | phosphatidic acid phosphatase type 2B | Ppap2b | 1.27 |
| 1431049_at | amyotrophic lateral sclerosis 2 (juvenile) chromosome region, ca [symbol] | Als2cr15 | 1.27 |
| 1451972_at | glucocorticoid induced transcript 1 | Glcci1 | 1.27 |
| 1452771_s_at | acyl-CoA synthetase long-chain family member 3 | Acsl3 | 1.27 |
| 1416257_at | calpain 2 | Capn2 | 1.26 |
| 1421317_x_at | myeloblastosis oncogene | Myb | 1.26 |
| 1428259_at | RIKEN cDNA 2310075M15 gene | 2310075M15Rik | 1.26 |
| 1431300_at | SH3-domain GRB2-like (endophilin) interacting protein 1 | Sgip1 | 1.26 |
| 1450194_a_at | myeloblastosis oncogene | Myb | 1.26 |
| 1454764_s_at | solute carrier family 38, member 1 | Slc38a1 | 1.26 |
| 1455549_at | SEC14 and spectrin domains 1 | Sestd1 | 1.26 |
| 1434496_at | polo-like kinase 3 (Drosophila) | Plk3 | 1.25 |
| 1452365_at | RIKEN cDNA 4732435N03 gene | 4732435N03Rik | 1.25 |
| 1417686_at | lectin, galactose binding, soluble 12 | Lgals12 | 1.24 |
| 1418209_a_at | profilin 2 | Pfn2 | 1.24 |
| 1434429_at | synaptotagmin 14-like | Syt14l | 1.24 |
| 1441463_at | RIKEN cDNA C230079D11 gene | C230079D11Rik | 1.24 |
| 1455250_at | SH3-domain binding protein 4 | Sh3bp4 | 1.24 |
| 1436508_at | RIKEN cDNA 2410014A08 gene | 2410014A08Rik | 1.23 |
| 1450857_a_at | procollagen, type I, alpha 2 | Col1a2 | 1.23 |
| 1426317_at | StAR-related lipid transfer (START) domain containing 6 | Stard6 | 1.22 |
| 1436509_at | RIKEN cDNA 2410014A08 gene | 2410014A08Rik | 1.22 |
| 1441927_at | synaptotagmin 7 | Syt7 | 1.22 |
| 1452214_at | SKI-like | Skil | 1.22 |
| 1454576_at | RIKEN cDNA A230102021 gene | A230102O21Rik | 1.22 |
| 1455818_at | RIKEN cDNA 4930427A07 gene | 4930427A07Rik | 1.22 |
| 1456874_at | fibronectin leucine rich transmembrane protein 2 | Flrt2 | 1.22 |
| 1458140_at | slit homolog 2 (Drosophila) | Slit2 | 1.22 |
| 1426218_at | glucocorticoid induced transcript 1 | Glcci1 | 1.21 |
| 1433043_at | chondroitin sulfate proteoglycan 2 | Cspg2 | 1.21 |
| 1450860_at | leucine aminopeptidase 3 | Lap3 | 1.21 |
| 1452107_s_at | nephronectin | Npnt | 1.21 |
| 1459868_x_at | interleukin 11 receptor, alpha chain 1 | Il11ra1 | 1.21 |
| 1417420_at | cyclin D1 | Ccnd1 | 1.20 |
| 1421052_a_at | spermine synthase | Sms | 1.20 |
| 1430934_at | RIKEN cDNA 2810043G22 gene | 2810043G22Rik | 1.20 |
| 1445178_at | SH3 multiple domains 2 | Sh3md2 | 1.20 |
| 1449630_s_at | MAP/microtubule affinity-regulating kinase 1 | Mark1 | 1.20 |
| 1454877_at | SERTA domain containing 4 | Sertad4 | 1.20 |
| 1417311_at | cysteine rich protein 2 | Crip2 | 1.19 |
| 1425942_a_at | glycoprotein m6b | Gpm6b | 1.19 |
| 1442845_at | RIKEN cDNA C130075A20 gene | C130075A20Rik | 1.19 |
| 1443589_at | DNA segment, Chr X, ERATO Doi 242, expressed | DXErtd242e | 1.19 |
| 1446850_at | Phosphatidic acid phosphatase type 2B | Ppap2b | 1.19 |
| 1448569_at | CD8 antigen, beta chain | Cd8b | 1.19 |
| 1450780_s_at | high mobility group AT-hook 2 | Hmga2 | 1.19 |
| 1457113_at | nuclear autoantigenic sperm protein (histone-binding) | Nasp | 1.19 |
| 1416489_at | phosphatidylinositol 4-kinase type 2 beta | Pi4k2b | 1.18 |
| 1435363_at | pleckstrin homology domain containing, family G (with RhoGef d [symbol] | Plekhg1 | 1.18 |
| 1441038_at | Utrophin | Utrn | 1.18 |
| 1452666_a_at | transmembrane and coiled-coil domains 2 | Tmcc2 | 1.18 |
| 1454268_a_at | cytochrome b-245, alpha polypeptide | Cyba | 1.18 |
| 1455214_at | microphthalmia-associated transcription factor | Mitf | 1.18 |
| 1440952_at | MAD homolog 7 (Drosophila) | Smad7 | 1.17 |
| 1444253_at | a disintegrin-like and metalloprotease (reprolysin type) with thror [symbol] | Adamts18 | 1.17 |
| 1451679_at | RIKEN cDNA 6530401D17 gene | 6530401D17Rik | 1.17 |
| 1455437_at | cDNA sequence BC033915 | BC033915 | 1.17 |
| 1423680_at | fatty acid desaturase 1 | Fads1 | 1.16 |
| 1426241_a_at | sex comb on midleg homolog 1 | Scmh1 | 1.16 |
| 1440513_at | expressed sequence C80258 | C80258 | 1.16 |
| 1445918_at | transmembrane protein 2 | Tmem2 | 1.16 |
| 1449455_at | hemopoietic cell kinase | Hck | 1.16 |
| 1458942_at | RIKEN cDNA C230037E05 gene | C230037E05Rik | 1.16 |
| 1460693_a_at | procollagen, type IX, alpha 3 | Col9a3 | 1.16 |
| 1416579_a_at | tumor-associated calcium signal transducer 1 | Tacstd1 | 1.15 |
| 1416953_at | connective tissue growth factor | Ctgf | 1.15 |
| 1424824_at | RIKEN cDNA 9630044O09 gene | 9630044O09Rik | 1.15 |
| 1433491_at | erythrocyte protein band 4.1-like 2 | Epb4.1l2 | 1.15 |
| 1436977_at | Transcribed sequences | NA | 1.15 |
| 1440284_at | Transcribed sequences | NA | 1.15 |
| 1452226_at | regulator of chromosome condensation 2 | Rcc2 | 1.15 |
| 1454701_at | RIKEN cDNA 4930503L19 gene | 4930503L19Rik | 1.15 |
| 1456072_at | protein phosphatase 1, regulatory (inhibitor) subunit 9A | Ppp1r9a | 1.15 |
| 1419798_at | RIKEN cDNA 2610019E17 gene | 2610019E17Rik | 1.14 |
| 1427028_at | leucine-rich repeat-containing G protein-coupled receptor 6 | Lgr6 | 1.14 |
| 1431226_a_at | fibronectin type III domain containing 4 | Fndc4 | 1.14 |
| 1434307_at | transmembrane protein 64 | Tmem64 | 1.14 |
| 1438783_at | Transmembrane, prostate androgen induced RNA | Tmepai | 1.14 |
| 1442175_at | RIKEN cDNA C030027H14 gene | C030027H14Rik | 1.14 |
| 1447043_at | V-erb-a erythroblastic leukemia viral oncogene homolog 4 (avia [symbol] | Erbb4 | 1.14 |
| 1451516_at | Ras homolog enriched in brain like 1 | Rhebl1 | 1.14 |
| 1456648_at | Mannosidase 1, alpha | Man1a | 1.14 |
| 1459144_at | Fibronectin type III domain containing 3a | Fndc3 | 1.14 |
| 1429896_at | RIKEN cDNA 5830408B19 gene | 5830408B19Rik | 1.13 |
| 1433894_at | expressed sequence AI591476 | AI591476 | 1.13 |
| 1437785_at | a disintegrin-like and metalloprotease (reprolysin type) with thro [symbol] | Adamts9 | 1.13 |
| 1446461_at | SRY-box containing gene 5 | Sox5 | 1.13 |
| 1453416_at | growth arrest-specific 2 like 3 | Gas2l3 | 1.13 |
| 1423499_at | synuclein, alpha interacting protein (synphilin) | Sncaip | 1.12 |
| 1424367_a_at | homer homolog 2 (Drosophila) | Homer2 | 1.12 |
| 1430033_at | RIKEN cDNA 5330431K02 gene | 5330431K02Rik | 1.12 |
| 1437059_at | SRY-box containing gene 21 | Sox21 | 1.12 |
| 1437467_at | activated leukocyte cell adhesion molecule | Alcam | 1.12 |
| 1439568_at | gene regulated by estrogen in breast cancer protein | Greb1 | 1.12 |
| 1446653_at | Transcribed sequences | NA | 1.12 |
| 1448919_at | CD302 antigen | Cd302 | 1.12 |
| 1430352_at | RIKEN cDNA 8430417A20 gene | 8430417A20Rik | 1.11 |
| 1431402_at | kin of IRRE like 3 (Drosophila) | Kirrel3 | 1.11 |
| 1433590_at | hect domain and RLD 3 | Herc3 | 1.11 |
| 1435083_at | cortexin | Ctxn | 1.11 |
| 1435176_a_at | inhibitor of DNA binding 2 | Id2 | 1.11 |
| 1459493_at | SET binding protein 1 (Setbp1), mRNA | Setbp1 | 1.11 |
| 1430083_at | RIKEN cDNA 2610307P16 gene | 2610307P16Rik | 1.10 |
| 1440739_at | vascular endothelial growth factor C | Vegfc | 1.10 |
| 1420411_a_at | phosphatidylinositol 4-kinase type 2 beta | Pi4k2b | 1.09 |
| 1422865_at | runt related transcription factor 1 | Runx1 | 1.09 |
| 1434645_at | RIKEN cDNA C530008M17 gene | C530008M17Rik | 1.09 |
| 1434797_at | RIKEN cDNA 6720469N11 gene | 6720469N11Rik | 1.09 |
| 1435050_at | RIKEN cDNA B930094H20 gene | B930094H20Rik | 1.09 |
| 1435977_at | hepatoma-derived growth factor, related protein 3 | Hdgfrp3 | 1.09 |
| 1438866_at | glutamate receptor ionotropic, NMDA3A | Grin3a | 1.09 |
| 1446897_at | Transcribed sequences | NA | 1.09 |
| 1448024_at | RIKEN cDNA B430320C24 gene | B430320C24Rik | 1.09 |
| 1458241_at | RIKEN cDNA 9230105E05 gene | 9230105E05Rik | 1.09 |
| 1419829_a_at | Transcribed sequence with weak similarity to protein prf: 201819 [symbol] | NA | 1.08 |
| 1435387_at | expressed sequence AI505012 | AI505012 | 1.08 |
| 1447818_x_at | Ras homolog enriched in brain like 1 | Rhebl1 | 1.08 |
| 1449058_at | GLI-Kruppel family member GLI1 | Gli1 | 1.08 |
| 1449471_at | potassium large conductance calcium-activated channel, subfam [symbol] | Kcnmb4 | 1.08 |
| 1456377_x_at | RIKEN cDNA 0610025L06 gene | 0610025L06Rik | 1.08 |
| 1460559_at | ankyrin repeat domain 25 | Ankrd25 | 1.08 |
| 1417312_at | dickkopf homolog 3 (Xenopus laevis) | Dkk3 | 1.07 |
| 1418711_at | platelet derived growth factor, alpha | Pdgfa | 1.07 |
| 1420342_at | ganglioside-induced differentiation-associated-protein 10 | Gdap10 | 1.07 |
| 1422462_at | ubiquitin-conjugating enzyme E2T (putative) | Ube2t | 1.07 |
| 1424250_a_at | Rho guanine nucleotide exchange factor (GEF) 3 | Arhgef3 | 1.07 |
| 1426934_at | NHS-like 1 | Nhsl1 | 1.07 |
| 1433531_at | acyl-CoA synthetase long-chain family member 4 | Acsl4 | 1.07 |
| 1434982_at | Transcribed sequence with weak similarity to protein pir: S07330 [symbol] | NA | 1.07 |
| 1440067_at | Neural cell adhesion molecule 1 | Ncam1 | 1.07 |
| 1418884_x_at | tubulin, alpha 1 | Tuba1 | 1.06 |
| 1424051_at | procollagen, type IV, alpha 2 | Col4a2 | 1.06 |
| 1424950_at | SRY-box containing gene 9 | Sox9 | 1.06 |
| 1425452_s_at | protein tyrosine phosphatase, receptor type, J /// expressed seq [symbol] | Ptprj /// AW125753 | 1.06 |
| 1425510_at | MAP/microtubule affinity-regulating kinase 1 | Mark1 | 1.06 |
| 1426541_a_at | RIKEN cDNA 2310067E08 gene | 2310067E08Rik | 1.06 |
| 1434592_at | solute carrier family 16 (monocarboxylic acid transporters), mem [symbol] | Slc16a10 | 1.06 |
| 1435184_at | RIKEN cDNA B430320C24 gene | B430320C24Rik | 1.06 |
| 1452205_x_at | T-cell receptor beta, variable 13 | Tcrb-V13 | 1.06 |
| 1452740_at | myosin heavy chain 10, non-muscle | Myh10 | 1.06 |
| 1457256_x_at | patched homolog 2 | Ptch2 | 1.06 |
| 1416505_at | nuclear receptor subfamily 4, group A, member 1 | Nr4a1 | 1.05 |
| 1417101_at | heat shock protein 2 | Hspa2 | 1.05 |
| 1417558_at | Fyn proto-oncogene | Fyn | 1.05 |
| 1421943_at | transforming growth factor alpha | Tgfa | 1.05 |
| 1422771_at | MAD homolog 6 (Drosophila) | Smad6 | 1.05 |
| 1427683_at | early growth response 2 | Egr2 | 1.05 |
| 1429114_at | SEC14 and spectrin domains 1 | Sestd1 | 1.05 |
| 1435913_at | cDNA sequence BC038881 | BC038881 | 1.05 |
| 1454671_at | insulin induced gene 1 | Insig1 | 1.05 |
| 1460627_at | RIKEN cDNA D130067I03 gene | D130067I03Rik | 1.05 |
| 1417612_at | immediate early response 5 | Ier5 | 1.04 |
| 1417978_at | eukaryotic translation initiation factor 4E member 3 | Eif4e3 | 1.04 |
| 1424594_at | lectin, galactose binding, soluble 7 | Lgals7 | 1.04 |
| 1434700_at | RIKEN cDNA 6030408C04 gene | 6030408C04Rik | 1.04 |
| 1435308_at | fucosyltransferase 9 | Fut9 | 1.04 |
| 1444519_at | G protein-coupled receptor 49 | Gpr49 | 1.04 |
| 1448416_at | matrix gamma-carboxyglutamate (gla) protein | Mglap | 1.04 |
| 1455820_x_at | scavenger receptor class B, member 1 | Scarb1 | 1.04 |
| 1458575_at | SET binding protein 1 (Setbp1), mRNA | Setbp1 | 1.04 |
| 1415806_at | plasminogen activator, tissue | Plat | 1.03 |
| 1434008_at | sodium channel, type IV, beta polypeptide | Scn4b | 1.03 |
| 1435461_at | membrane associated guanylate kinase, WW and PDZ domain [symbol] | Magi3 | 1.03 |
| 1436368_at | solute carrier family 16 (monocarboxylic acid transporters), mem [symbol] | Slc16a10 | 1.03 |
| 1439926_at | hypothetical protein 4632417D23 | 4632417D23 | 1.03 |
| 1455881_at | immediate early response 5-like | Ier5l | 1.03 |
| 1415964_at | stearoyl-Coenzyme A desaturase 1 | Scd1 | 1.02 |
| 1417688_at | cDNA sequence BC004044 | BC004044 | 1.02 |
| 1417749_a_at | tight junction protein 1 | Tjp1 | 1.02 |
| 1432746_at | RIKEN cDNA 6030442H21 gene | 6030442H21Rik | 1.02 |
| 1437540_at | mucolipin 3 | Mcoln3 | 1.02 |
| 1444583_at | Nuclear autoantigenic sperm protein (histone-binding) | Nasp | 1.02 |
| 1448509_at | SH3-domain GRB2-like (endophilin) interacting protein 1 | Sgip1 | 1.02 |
| 1451152_a_at | ATPase, Na+/K+ transporting, beta 1 polypeptide | Atp1b1 | 1.02 |
| 1455149_at | SH3 multiple domains 2 | Sh3md2 | 1.02 |
| 1456475_s_at | protein kinase, cAMP dependent regulatory, type II beta | Prkar2b | 1.02 |
| 1416326_at | cysteine-rich protein 1 (intestinal) | Crip1 | 1.01 |
| 1417928_at | PDZ and LIM domain 4 | Pdlim4 | 1.01 |
| 1419593_at | gene regulated by estrogen in breast cancer protein | Greb1 | 1.01 |
| 1433571_at | serine incorporator 5 | Serinc5 | 1.01 |
| 1433770_at | dihydropyrimidinase-like 2 | Dpysl2 | 1.01 |
| 1441995_at | Neural cell adhesion molecule 1 | Ncam1 | 1.01 |
| 1448908_at | phosphatidic acid phosphatase type 2B | Ppap2b | 1.01 |
| 1455687_at | intestinal cell kinase | Ick | 1.01 |
| 1455980_a_at | PREDICTED: similar to growth arrest-specific 2 like 3 [Mus mus [symbol] | Gas2l3 | 1.01 |
| 1457229_at | G-protein coupled receptor 173 | Gpr173 | 1.01 |
| 1459054_at | cDNA sequence BC035954 | BC035954 | 1.01 |
| 1426712_at | solute carrier family 6 (neurotransmitter transporter), member 15 | Slc6a15 | 1.00 |
| 1428749_at | Dmx-like 2 | Dmxl2 | 1.00 |
| 1437894_at | prospero-related homeobox 1 | Prox1 | 1.00 |
| 1438664_at | protein kinase, cAMP dependent regulatory, type II beta | Prkar2b | 1.00 |
| 1446326_at | procollagen, type I, alpha 2 | Col1a2 | 1.00 |
| 1449198_a_at | sialyltransferase 9 (CMP-NeuAc:lactosylceramide alpha-2,3-sial [symbol] | Siat9 | 1.00 |
| 1453523_at | RIKEN cDNA A030006P16 gene | A030006P16Rik | 1.00 |
| 1419161_a_at | NADPH oxidase 4 | Nox4 | −1.00 |
| 1419309_at | podoplanin | Pdpn | −1.00 |
| 1426584_a_at | sorbitol dehydrogenase 1 | Sdh1 | −1.00 |
| 1428549_at | coiled-coil domain containing 3 | Ccdc3 | −1.00 |
| 1429021_at | Eph receptor A4 | Epha4 | −1.00 |
| 1429581_at | acyl-Coenzyme A dehydrogenase family, member 9 | Acad9 | −1.00 |
| 1430191_at | RIKEN cDNA 9130004J05 gene | 9130004J05Rik | −1.00 |
| 1439731_at | RIKEN cDNA E130309F12 gene | E130309F12Rik | −1.00 |
| 1445897_s_at | interferon-induced protein 35 | Ifi35 | −1.00 |
| 1451243_at | arginyl aminopeptidase (aminopeptidase B) | Rnpep | −1.00 |
| 1416137_at | annexin A7 | Anxa7 | −1.01 |
| 1416401_at | kangai 1 (suppression of tumorigenicity 6, prostate) | Kai1 | −1.01 |
| 1417701_at | protein phosphatase 1, regulatory (inhibitor) subunit 14c | Ppp1r14c | −1.01 |
| 1422470_at | BCL2/adenovirus E1B 19 kDa-interacting protein 1, NIP3 | Bnip3 | −1.01 |
| 1424172_at | hydroxyacyl glutathione hydrolase | Hagh | −1.01 |
| 1427510_at | guanine nucleotide binding protein, alpha inhibiting 1 | Gnai1 | −1.01 |
| 1430286_s_at | protein phosphatase 1, regulatory (inhibitor) subunit 14c | Ppp1r14c | −1.01 |
| 1449256_a_at | RAB11a, member RAS oncogene family | Rab11a | −1.01 |
| 1452434_s_at | DiGeorge syndrome critical region gene 6 | Dgcr6 | −1.01 |
| 1452519_a_at | zinc finger protein 36 | Zfp36 | −1.01 |
| 1453261_at | RIKEN cDNA 2610035D17 gene | 2610035D17Rik | −1.01 |
| 1416021_a_at | fatty acid binding protein 5, epidermal | Fabp5 | −1.02 |
| 1417195_at | DNA segment, Chr 8, ERATO Doi 594, expressed | D8Ertd594e | −1.02 |
| 1417649_at | cyclin-dependent kinase inhibitor 1C (P57) | Cdkn1c | −1.02 |
| 1420150_at | splA/ryanodine receptor domain and SOCS box containing 1 | Spsb1 | −1.02 |
| 1422743_at | phosphorylase kinase alpha 1 | Phka1 | −1.02 |
| 1423420_at | adrenergic receptor, beta 1 | Adrb1 | −1.02 |
| 1423909_at | RIKEN cDNA 0610011I04 gene | 0610011I04Rik | −1.02 |
| 1426873_s_at | junction plakoglobin | Jup | −1.02 |
| 1438764_at | annexin A7 | Anxa7 | −1.02 |
| 1419052_at | OVO homolog-like 1 (Drosophila) | Ovol1 | −1.03 |
| 1435205_at | transcription factor AP-2, epsilon | Tcfap2e | −1.03 |
| 1435571_at | RIKEN cDNA A530065I17 gene | A530065I17Rik | −1.03 |
| 1438093_x_at | diazepam binding inhibitor | Dbi | −1.03 |
| 1442606_at | dynamin binding protein | Dnmbp | −1.03 |
| 1455994_x_at | elongation of very long chain fatty acids (FEN1/Elo2, SUR4/Elo3 [symbol] | Elovl1 | −1.03 |
| 1457883_at | Adult male aorta and vein cDNA, RIKEN full-length enriched libr [symbol] | NA | −1.03 |
| 1459948_at | Growth hormone receptor | Ghr | −1.03 |
| 1418517_at | Iroquois related homeobox 3 (Drosophila) | Irx3 | −1.04 |
| 1421106_at | jagged 1 | Jag1 | −1.04 |
| 1424530_at | SEC14-like 2 (S. cerevisiae) | Sec14l2 | −1.04 |
| 1425645_s_at | cytochrome P450, family 2, subfamily b, polypeptide 20 | Cyp2b20 | −1.04 |
| 1430483_a_at | RIKEN cDNA 2310042N02 gene | 2310042N02Rik | −1.04 |
| 1430655_at | RIKEN cDNA 4631405K08 gene | 4631405K08Rik | −1.04 |
| 1441547_at | Nuclear factor I/A | Nfia | −1.04 |
| 1446281_at | RIKEN cDNA C530008M07 gene | C530008M07Rik | −1.04 |
| 1449852_a_at | EH-domain containing 4 | Ehd4 | −1.04 |
| 1423494_at | RIKEN cDNA 2310042E22 gene | 2310042E22Rik | −1.05 |
| 1428400_at | RIKEN cDNA 2200002K05 gene | 2200002K05Rik | −1.05 |
| 1430724_at | abhydrolase domain containing 9 | Abhd9 | −1.05 |
| 1432018_at | achaete-scute complex homolog-like 2 (Drosophila) | Ascl2 | −1.05 |
| 1433943_at | RIKEN cDNA 4930463G05 gene | 4930463G05Rik | −1.05 |
| 1448529_at | thrombomodulin | Thbd | −1.05 |
| 1450034_at | signal transducer and activator of transcription 1 | Stat1 | −1.05 |
| 1451210_at | phosphatidic acid phosphatase type 2c | Ppap2c | −1.05 |
| 1417047_at | prominin 2 | Prom2 | −1.06 |
| 1417196_s_at | DNA segment, Chr 8, ERATO Doi 594, expressed | D8Ertd594e | −1.06 |
| 1425603_at | RIKEN cDNA 0610011I04 gene | 0610011I04Rik | −1.06 |
| 1448194_a_at | H19 fetal liver mRNA | H19 | −1.06 |
| 1453511_at | RIKEN cDNA 2310007B03 gene | 2310007B03Rik | −1.06 |
| 1458578_at | RNA binding motif, single stranded interacting protein 1, mRNA | Rbms1 | −1.06 |
| 1417301_at | frizzled homolog 6 (Drosophila) | Fzd6 | −1.07 |
| 1422784_at | keratin complex 2, basic, gene 6a | Krt2-6a | −1.07 |
| 1424308_at | solute carrier family 24 (sodium/potassium/calcium exchanger), | Slc24a3 | −1.07 |
| 1425918_at | EGL nine homolog 3 (C. elegans) | Egln3 | −1.07 |
| 1438937_x_at | angiogenin, ribonuclease A family, member 1 | Ang1 | −1.07 |
| 1448985_at | dual specificity phosphatase 22 | Dusp22 | −1.07 |
| 1450626_at | mannosidase, beta A, lysosomal | Manba | −1.07 |
| 1453593_at | RIKEN cDNA 1700110N18 gene | 1700110N18Rik | −1.07 |
| 1416431_at | tubulin, beta 6 | Tubb6 | −1.08 |
| 1417395_at | Kruppel-like factor 4 (gut) | Klf4 | −1.08 |
| 1420614_at | t-complex-associated-testis-expressed 1-like | Tcte1l | −1.08 |
| 1424400_a_at | formyltetrahydrofolate dehydrogenase | Fthfd | −1.08 |
| 1428450_at | RIKEN cDNA 2610034B18 gene | 2610034B18Rik | −1.08 |
| 1437057_at | EGF-like-domain, multiple 3 | Egfl3 | −1.08 |
| 1437245_at | RIKEN cDNA 9930117H01 gene | 9930117H01Rik | −1.08 |
| 1439757_s_at | Eph receptor A4 | Epha4 | −1.08 |
| 1440693_at | RIKEN cDNA 2410003B16 gene | 2410003B16Rik | −1.08 |
| 1443720_s_at | bone morphogenetic protein receptor, type 1B | Bmpr1b | −1.08 |
| 1445929_at | Potassium channel, subfamily K, member 2 | Kcnk2 | −1.08 |
| 1415850_at | RAS p21 protein activator 3 | Rasa3 | −1.09 |
| 1417777_at | leukotriene B4 12-hydroxydehydrogenase | Ltb4dh | −1.09 |
| 1418483_a_at | glycoprotein galactosyltransferase alpha 1, 3 | Ggta1 | −1.09 |
| 1418648_at | EGL nine homolog 3 (C. elegans) | Egln3 | −1.09 |
| 1420498_a_at | disabled homolog 2 (Drosophila) | Dab2 | −1.09 |
| 1422293_a_at | potassium channel tetramerisation domain containing 1 | Kctd1 | −1.09 |
| 1423126_at | ATPase, Na+/K+ transporting, beta 3 polypeptide | Atp1b3 | −1.09 |
| 1424507_at | Ras and Rab interactor 1 | Rin1 | −1.09 |
| 1434440_at | guanine nucleotide binding protein, alpha inhibiting 1 | Gnai1 | −1.09 |
| 1434725_at | RIKEN cDNA 4921521N14 gene | 4921521N14Rik | −1.09 |
| 1436346_at | CD109 antigen | Cd109 | −1.09 |
| 1440283_at | RIKEN cDNA 1810059H22 gene | 1810059H22Rik | −1.09 |
| 1441163_at | mediator of RNA polymerase II transcription, subunit 12 homolog [symbol] | Med12l | −1.09 |
| 1447655_x_at | SRY-box containing gene 6 | Sox6 | −1.09 |
| 1448954_at | nuclear receptor interacting protein 3 | Nrip3 | −1.09 |
| 1449929_at | t-complex-associated-testis-expressed 1-like | Tcte1l | −1.09 |
| 1450947_at | RIKEN cDNA 2610528J11 gene | 2610528J11Rik | −1.09 |
| 1416528_at | SH3 domain binding glutamic acid-rich protein-like 3 | Sh3bgrl3 | −1.10 |
| 1424030_at | grainyhead-like 1 (Drosophila) | Grhl1 | −1.10 |
| 1424942_a_at | myelocytomatosis oncogene | Myc | −1.10 |
| 1429284_at | RIKEN cDNA 8430436F23 gene | 8430436F23Rik | −1.10 |
| 1434534_at | NA | NA | −1.10 |
| 1437874_s_at | hexosaminidase B | Hexb | −1.10 |
| 1444974_at | expressed sequence AU023617 | AU023617 | −1.10 |
| 1451594_s_at | serine (or cysteine) proteinase inhibitor, clade B, member 6c | Serpinb6c | −1.10 |
| 1416564_at | SRY-box containing gene 7 | Sox7 | −1.11 |
| 1427677_a_at | SRY-box containing gene 6 | Sox6 | −1.11 |
| 1436651_at | Transcribed sequences | NA | −1.11 |
| 1438882_at | Rho GTPase activating protein 18 | Arhgap18 | −1.11 |
| 1439352_at | tripartite motif protein 7 | Trim7 | −1.11 |
| 1454862_at | pleckstrin homology-like domain, family B, member 2 | Phldb2 | −1.11 |
| 1417122_at | vav 3 oncogene | Vav3 | −1.12 |
| 1419572_a_at | ATP-binding cassette, sub-family D (ALD), member 4 | Abcd4 | −1.12 |
| 1424303_at | expressed sequence AV216087 | AV216087 | −1.12 |
| 1425108_a_at | cDNA sequence BC004728 | B0004728 | −1.12 |
| 1426908_at | UDP-N-acetyl-alpha-D-galactosamine: polypeptide N-acetylgala [symbol] | Galnt7 | −1.12 |
| 1441153_at | Utrophin | Utrn | −1.12 |
| 1443997_at | RIKEN cDNA C130040D06 gene | C130040D06Rik | −1.12 |
| 1416008_at | special AT-rich sequence binding protein 1 | Satb1 | −1.13 |
| 1417813_at | inhibitor of kappaB kinase epsilon | Ikbke | −1.13 |
| 1421163_a_at | nuclear factor I/A | Nfia | −1.13 |
| 1434070_at | jagged 1 | Jag1 | −1.13 |
| 1437302_at | adrenergic receptor, beta 2 | Adrb2 | −1.13 |
| 1438183_x_at | sorbitol dehydrogenase 1 | Sdh1 | −1.13 |
| 1440491_at | solute carrier family 1 (glial high affinity glutamate transporter), [symbol] | Slc1a3 | −1.13 |
| 1448359_a_at | hypoxia induced gene 1 | Hig1 | −1.13 |
| 1451446_at | anthrax toxin receptor 1 | Antxr1 | −1.13 |
| 1451775_s_at | interleukin 13 receptor, alpha 1 | Il13ra1 | −1.13 |
| 1416855_at | growth arrest specific 1 | Gas1 | −1.14 |
| 1417566_at | abhydrolase domain containing 5 | Abhd5 | −1.14 |
| 1417639_at | solute carrier family 22 (organic cation transporter), member 4 | Slc22a4 | −1.14 |
| 1420915_at | signal transducer and activator of transcription 1 | Stat1 | −1.14 |
| 1422678_at | diacylglycerol O-acyltransferase 2 | Dgat2 | −1.14 |
| 1423824_at | RIKEN cDNA 5031439A09 gene | 5031439A09Rik | −1.14 |
| 1426952_at | Rho GTPase activating protein 18 | Arhgap18 | −1.14 |
| 1433819_s_at | 1-acylglycerol-3-phosphate O-acyltransferase 3 | Agpat3 | −1.14 |
| 1434486_x_at | UDP-glucose pyrophosphorylase 2 | Ugp2 | −1.14 |
| 1444479_at | Transcribed sequences | NA | −1.14 |
| 1447173_at | NA | NA | −1.14 |
| 1450618_a_at | small proline-rich protein 2A | Sprr2a | −1.14 |
| 1423062_at | insulin-like growth factor binding protein 3 | Igfbp3 | −1.15 |
| 1435842_at | RIKEN cDNA 1110038O08 gene | 1110038O08Rik | −1.15 |
| 1437458_x_at | clusterin | Clu | −1.15 |
| 1437932_a_at | claudin 1 | Cldn1 | −1.15 |
| 1448312_at | proprotein convertase subtilisin/kexin type 2 | Pcsk2 | −1.15 |
| 1451690_a_at | poliovirus receptor-related 4 | Pvrl4 | −1.15 |
| 1453072_at | G protein-coupled receptor 160 | Gpr160 | −1.15 |
| 1456321_at | RIKEN cDNA 3830408G10 gene | 3830408G10Rik | −1.15 |
| 1415943_at | syndecan 1 | Sdc1 | −1.16 |
| 1416481_s_at | hypoxia induced gene 1 | Hig1 | −1.16 |
| 1421217_a_at | lectin, galactose binding, soluble 9 | Lgals9 | −1.16 |
| 1421365_at | follistatin | Fst | −1.16 |
| 1423844_s_at | cystathionine beta-synthase | Cbs | −1.16 |
| 1437279_x_at | syndecan 1 | Sdc1 | −1.16 |
| 1439148_a_at | phosphofructokinase, liver, B-type | Pfkl | −1.16 |
| 1439847_s_at | Kruppel-like factor 12 | Klf12 | −1.16 |
| 1444274_at | Transcribed sequence with moderate similarity to protein pir: S12 [symbol] | NA | −1.16 |
| 1450976_at | N-myc downstream regulated gene 1 | Ndrg1 | −1.16 |
| 1451546_s_at | transmembrane protein 40 | Tmem40 | −1.16 |
| 1455872_at | hypothetical protein A030013D21 | A030013D21 | −1.16 |
| 1460236_at | kallikrein 10 | Klk10 | −1.16 |
| 1422941_at | wingless-related MMTV integration site 16 | Wnt16 | −1.17 |
| 1426604_at | ribonuclease L (2′,5′-oligoisoadenylate synthetase-dependent) | Rnasel | −1.17 |
| 1426911_at | desmocollin 2 | Dsc2 | −1.17 |
| 1431777_a_at | high mobility group nucleosomal binding domain 3 | Hmgn3 | −1.17 |
| 1433266_at | RIKEN cDNA 2810416A17 gene | 2810416A17Rik | −1.17 |
| 1441971_at | Transcribed sequences | NA | −1.17 |
| 1451742_a_at | UDP-glucose pyrophosphorylase 2 | Ugp2 | −1.17 |
| 1452092_at | RIKEN cDNA 4631426J05 gene | 4631426J05Rik | −1.17 |
| 1459009_at | Utrophin | Utrn | −1.17 |
| 1417143_at | endothelial differentiation, lysophosphatidic acid G-protein-coup [symbol] | Edg2 | −1.18 |
| 1438251_x_at | protease, serine, 11 (lgf binding) | Prss11 | −1.18 |
| 1441242_at | Dipeptidylpeptidase 4 | Dpp4 | −1.18 |
| 1442348_at | 2 days pregnant adult female oviduct cDNA, RlKEN full-length e [symbol] | NA | −1.18 |
| 1445710_x_at | RIKEN cDNA 1110051B16 gene | 1110051B16Rik | −1.18 |
| 1455915_at | UDP-N-acetyl-alpha-D-galactosamine:polypeptide N-acetylgala [symbol] | Galnt4 | −1.18 |
| 1416455_a_at | crystallin, alpha B | Cryab | −1.19 |
| 1418135_at | AF4/FMR2 family, member 1 | Aff1 | −1.19 |
| 1418497_at | fibroblast growth factor 13 | Fgf13 | −1.19 |
| 1420822_s_at | sphingosine-1-phosphate phosphatase 1 | Sgpp1 | −1.19 |
| 1425560_a_at | S100 calcium binding protein A16 | S100a16 | −1.19 |
| 1434059_at | RIKEN cDNA B230312A22 gene | B230312A22Rik | −1.19 |
| 1446480_at | Adult male corpora quadrigemina cDNA, RIKEN full-length enric [symbol] | NA | −1.19 |
| 1415906_at | thymosin, beta 4, X chromosome | Tmsb4x | −1.20 |
| 1419663_at | osteoglycin | Ogn | −1.20 |
| 1421045_at | mannose receptor, C type 2 | Mrc2 | −1.20 |
| 1429582_at | BTB (POZ) domain containing 14A | Btbd14a | −1.20 |
| 1430114_at | RIKEN cDNA 5430420C16 gene | 5430420C16Rik | −1.20 |
| 1435059_at | development and differentiation enhancing | Ddef1 | −1.20 |
| 1435283_s_at | expressed sequence AI414343 | Al414343 | −1.20 |
| 1436994_a_at | histone 1, H1c | Hist1h1c | −1.20 |
| 1445421_at | Transcribed sequences | NA | −1.20 |
| 1447676_x_at | S100 calcium binding protein A16 | S100a16 | −1.20 |
| 1452138_a_at | angiotensin I converting enzyme (peptidyl-dipeptidase A) 2 | Ace2 | −1.20 |
| 1452388_at | heat shock protein 1A | Hspa1a | −1.20 |
| 1416101_a_at | histone 1, H1c | Hist1h1c | −1.21 |
| 1417795_at | cell adhesion molecule with homology to L1CAM | Chl1 | −1.21 |
| 1419239_at | zinc finger protein 54 | Zfp54 | −1.21 |
| 1425284_a_at | RAB27A, member RAS oncogene family | Rab27a | −1.21 |
| 1437868_at | cDNA sequence BC023892 | BC023892 | −1.21 |
| 1439184_s_at | thioredoxin-like 5 | Txnl5 | −1.21 |
| 1443906_at | decay accelerating factor 1 | Daf1 | −1.21 |
| 1448499_a_at | epoxide hydrolase 2, cytoplasmic | Ephx2 | −1.21 |
| 1458127_at | NA | NA | −1.21 |
| 1433971_at | calmodulin binding transcription activator 1 | Camta1 | −1.22 |
| 1439203_at | Special AT-rich sequence binding protein 1 | Satb1 | −1.22 |
| 1424359_at | 5-oxoprolinase (ATP-hydrolysing) | Oplah | −1.23 |
| 1432418_a_at | creatine kinase, mitochondrial 1, ubiquitous | Ckmt1 | −1.23 |
| 1434485_a_at | UDP-glucose pyrophosphorylase 2 | Ugp2 | −1.23 |
| 1447299_at | NA | NA | −1.23 |
| 1452160_at | TCDD-inducible poly(ADP-ribose) polymerase | Tiparp | −1.23 |
| 1416749_at | protease, serine, 11 (Igf binding) | Prss11 | −1.24 |
| 1418073_at | acyl-CoA thioesterase 9 | Acot9 | −1.24 |
| 1427477_at | transmembrane protease, serine 13 | Tmprss13 | −1.24 |
| 1436183_at | RIKEN cDNA 9830115L13 gene | 9830115L13Rik | −1.24 |
| 1436633_at | LOC380741 (LOC380741), mRNA | NA | −1.24 |
| 1437869_at | RIKEN cDNA 3222402P14 gene | 3222402P14Rik | −1.24 |
| 1438115_a_at | solute carrier family 9 (sodium/hydrogen exchanger), isoform 3 [symbol] | Slc9a3r1 | −1.24 |
| 1449773_s_at | growth arrest and DNA-damage-inducible 45 beta | Gadd45b | −1.24 |
| 1455372_at | cytoplasmic polyadenylation element binding protein 3 | Cpeb3 | −1.24 |
| 1456891_at | DENN/MADD domain containing 2C | Dennd2c | −1.24 |
| 1420821_at | sphingosine-1-phosphate phosphatase 1 | Sgpp1 | −1.25 |
| 1430567_at | serine protease inhibitor, Kazal type 5 | Spink5 | −1.25 |
| 1434575_at | erythrocyte protein band 4.1-like 1 | Epb4.1l1 | −1.25 |
| 1434738_at | threonyl-tRNA synthetase-like 2 | Tarsl2 | −1.25 |
| 1434802_s_at | neurotrophin 3 | Ntf3 | −1.25 |
| 1435354_at | potassium inwardly-rectifying channel, subfamily J, member 15 | Kcnj15 | −1.25 |
| 1437434_a_at | RIKEN cDNA 5031439A09 gene | 5031439A09Rik | −1.25 |
| 1437469_at | RIKEN cDNA A030007D23 gene | A030007D23Rik | −1.25 |
| 1438588_at | pleiomorphic adenoma gene-like 1 | Plagl1 | −1.25 |
| 1449968_s_at | acyl-Coenzyme A thioesterase 2, mitochondrial | Acate2 | −1.25 |
| 1453377_at | SH2 domain containing 4A | Sh2d4a | −1.25 |
| 1455786_at | RIKEN cDNA 2610036F08 gene | 2610036F08Rik | −1.25 |
| 1456003_a_at | solute carrier family 1 (glutamate/neutral amino acid transporter) | Slc1a4 | −1.25 |
| 1459823_at | EH-domain containing 2 | Ehd2 | −1.25 |
| 1416343_a_at | lysosomal membrane glycoprotein 2 | Lamp2 | −1.26 |
| 1418890_a_at | RAB3D, member RAS oncogene family | Rab3d | −1.26 |
| 1419133_at | envoplakin | Evpl | −1.26 |
| 1429637_at | RIKEN cDNA 2210419I08 gene | 2210419I08Rik | −1.26 |
| 1436122_at | Zinc finger protein 667 (Zfp667), mRNA | A830025F02Rik | −1.26 |
| 1436293_x_at | DNA segment, Chr 1, ERATO Doi 471, expressed | D1Ertd471e | −1.26 |
| 1448233_at | prion protein | Prnp | −1.26 |
| 1451201_s_at | ribonuclease/angiogenin inhibitor 1 | Rnh1 | −1.26 |
| 1455304_at | Similar to Munc13-3 (LOC235480), mRNA | NA | −1.26 |
| 1460351_at | S100 calcium binding protein A11 (calizzarin) | S100a11 | −1.26 |
| 1416921_x_at | aldolase 1, A isoform | Aldo1 | −1.27 |
| 1417580_s_at | selenium binding protein 1 | Selenbp1 | −1.27 |
| 1424877_a_at | aminolevulinate, delta-, dehydratase | Alad | −1.27 |
| 1433481_at | FK506 binding protein 14 | Fkbp14 | −1.27 |
| 1434815_a_at | mitogen-activated protein kinase-activated protein kinase 3 | Mapkapk3 | −1.27 |
| 1437058_at | EGF-like-domain, multiple 3 | Egfl3 | −1.27 |
| 1438116_x_at | solute carrier family 9 (sodium/hydrogen exchanger), isoform 3 [symbol] | Slc9a3r1 | −1.27 |
| 1443122_at | NA | NA | −1.27 |
| 1444441_at | Transcribed sequences | NA | −1.27 |
| 1451508_at | RIKEN cDNA 1700108L22 gene | 1700108L22Rik | −1.27 |
| 1418267_at | macrophage stimulating 1 (hepatocyte growth factor-like) | Mst1 | −1.28 |
| 1441793_at | Protein phosphatase 1, regulatory (inhibitor) subunit 11 | Ppp1r11 | −1.28 |
| 1448786_at | RIKEN cDNA 1100001H23 gene | 1100001H23Rik | −1.28 |
| 1449131_s_at | CD1d1 antigen | Cd1d1 | −1.28 |
| 1457358_at | Myeloid ecotropic viral integration site-related gene 1 | Mrg1 | −1.28 |
| 1416138_at | annexin A7 | Anxa7 | −1.29 |
| 1416194_at | cytochrome P450, family 4, subfamily b, polypeptide 1 | Cyp4b1 | −1.29 |
| 1422822_at | StAR-related lipid transfer (START) domain containing 5 | Stard5 | −1.29 |
| 1424096_at | keratin complex 2, basic, gene 5 | Krt2-5 | −1.29 |
| 1428922_at | RIKEN cDNA 1200009O22 gene | 1200009O22Rik | −1.29 |
| 1437665_at | Putative cytokine receptor family II soluble 1 precursor (CRF2-s [symbol] | Il22ra2 | −1.29 |
| 1439375_x_at | aldolase 1, A isoform | Aldo1 | −1.29 |
| 1448606_at | endothelial differentiation, lysophosphatidic acid G-protein-coup [symbol] | Edg2 | −1.29 |
| 1455521_at | Kruppel-like factor 12 | Klf12 | −1.29 |
| 1460407_at | Spi-B transcription factor (Spi-1/PU.1 related) | Spib | −1.29 |
| 1417335_at | sulfotransferase family, cytosolic, 2B, member 1 | Sult2b1 | −1.30 |
| 1423035_s_at | thioredoxin-like 5 | Txnl5 | −1.30 |
| 1427553_at | Clone IMAGE: 4190185, mRNA, partial cds | NA | −1.30 |
| 1438407_at | RIKEN cDNA 9330132E09 gene | 9330132E09Rik | −1.30 |
| 1445701_at | ATPase, Ca++ transporting, plasma membrane 4 (Atp2b4), mR [symbol] | Atp2b4 | −1.30 |
| 1460657_at | wingless related MMTV integration site 10a | Wnt10a | −1.30 |
| 1416554_at | PDZ and LIM domain 1 (elfin) | Pdlim1 | −1.31 |
| 1419401_at | ankyrin repeat and SOCS box-containing protein 13 | Asb13 | −1.31 |
| 1435435_at | cortactin binding protein 2 | Cortbp2 | −1.31 |
| 1437689_x_at | clusterin | Clu | −1.31 |
| 1448491_at | enoyl coenzyme A hydratase 1 peroxisomal | Ech1 | −1.31 |
| 1449130_at | CD1d1 antigen | Cd1d1 | −1.31 |
| 1452714_at | RIKEN cDNA 1200003E16 gene | 1200003E16Rik | −1.31 |
| 1420191_s_at | DNA segment, Chr 16, Brigham & Women's Genetics 1494 expr [symbol] | D16Bwg1494e | −1.32 |
| 1449036_at | ring finger protein 128 | Rnf128 | −1.32 |
| 1449770_x_at | DNA segment, Chr 16, Brigham & Women's Genetics 1494 expr [symbol] | D16Bwg1494e | −1.32 |
| 1451177_at | DnaJ (Hsp40) homolog, subfamily B, member 4 | Dnajb4 | −1.32 |
| 1425248_a_at | TYRO3 protein tyrosine kinase 3 | Tyro3 | −1.33 |
| 1447992_s_at | proprotein convertase subtilisin/kexin type 2 | Pcsk2 | −1.33 |
| 1452246_at | osteoclast stimulating factor 1 | Ostf1 | −1.33 |
| 1459749_s_at | FAT tumor suppressor homolog 4 (Drosophila) | Fat4 | −1.33 |
| 1416805_at | RIKEN cDNA 1110032E23 gene | 1110032E23Rik | −1.34 |
| 1418951_at | taxilin beta | Txlnb | −1.34 |
| 1420565_at | homeo box A1 | Hoxa1 | −1.34 |
| 1426721_s_at | TCDD-inducible poly(ADP-ribose) polymerase | Tiparp | −1.34 |
| 1430396_at | RIKEN cDNA 5730403I07 gene | 5730403I07Rik | −1.34 |
| 1433582_at | RIKEN cDNA 1190002N15 gene | 1190002N15Rik | −1.34 |
| 1439860_at | eukaryotic elongation factor-2 kinase | Eef2k | −1.34 |
| 1441774_at | Rab38, member of RAS oncogene family | Rab38 | −1.34 |
| 1445574_at | Transcribed sequences | NA | −1.34 |
| 1450726_at | N-acylsphingosine amidohydrolase 2 | Asah2 | −1.34 |
| 1455101_at | Clone IMAGE: 2647821, mRNA | NA | −1.34 |
| 1457533_at | Transcribed sequences | NA | −1.34 |
| 1417738_at | RAB25, member RAS oncogene family | Rab25 | −1.35 |
| 1420583_a_at | RAR-related orphan receptor alpha | Rora | −1.35 |
| 1423135_at | thymus cell antigen 1, theta | Thy1 | −1.35 |
| 1427228_at | RIKEN cDNA 2410003B16 gene | 2410003B16Rik | −1.35 |
| 1431057_a_at | protease, serine, 23 | Prss23 | −1.35 |
| 1438057_at | Transcribed sequence with moderate similarity to protein pdb: 1L [symbol] | NA | −1.35 |
| 1439429_x_at | deltex 2 homolog (Drosophila) | Dtx2 | −1.35 |
| 1442074_at | Transcribed sequences | NA | −1.35 |
| 1442791_x_at | RIKEN cDNA 6720407P12 gene | 6720407P12Rik | −1.35 |
| 1445558_at | NA | NA | −1.35 |
| 1450724_at | down-regulated by Ctnnb1, a | Drctnnb1a | −1.35 |
| 1415944_at | syndecan 1 | Sdc1 | −1.36 |
| 1419738_a_at | tropomyosin 2, beta | Tpm2 | −1.36 |
| 1422631_at | aryl-hydrocarbon receptor | Ahr | −1.36 |
| 1422637_at | Ras association (RalGDS/AF-6) domain family 5 | Rassf5 | −1.36 |
| 1422638_s_at | Ras association (RalGDS/AF-6) domain family 5 | Rassf5 | −1.36 |
| 1425658_at | CD109 antigen | Cd109 | −1.36 |
| 1426276_at | interferon induced with helicase C domain 1 | Ifih1 | −1.36 |
| 1432739_at | RIKEN cDNA 2900060K15 gene | 2900060K15Rik | −1.36 |
| 1434799_x_at | aldolase 1, A isoform | Aldo1 | −1.36 |
| 1436475_at | nuclear receptor subfamily 2, group F, member 2 | Nr2f2 | −1.36 |
| 1458706_at | Transcribed sequences | NA | −1.36 |
| 1452411_at | leucine rich repeat containing 1 | Lrrc1 | −1.37 |
| 1417823_at | glycine C-acetyltransferase (2-amino-3-ketobutyrate-coenzyme [symbol] | Gcat | −1.38 |
| 1418601_at | aldehyde dehydrogenase family 1, subfamily A7 | Aldh1a7 | −1.38 |
| 1422667_at | keratin complex 1, acidic, gene 15 | Krt1-15 | −1.38 |
| 1425002_at | cDNA sequence BC010462 | BC010462 | −1.38 |
| 1433604_x_at | aldolase 1, A isoform | Aldo1 | −1.38 |
| 1442560_at | Monoglyceride lipase | Mgll | −1.38 |
| 1455091_at | male-specific lethal-2 homolog (Drosophila) | Msl2 | −1.38 |
| 1418799_a_at | procollagen, type XVII, alpha 1 | Col17a1 | −1.39 |
| 1419091_a_at | annexin A2 | Anxa2 | −1.39 |
| 1433581_at | RIKEN cDNA 1190002N15 gene | 1190002N15Rik | −1.39 |
| 1435342_at | potassium inwardly-rectifying channel, subfamily K, member 6 | Kcnk6 | −1.39 |
| 1449459_s_at | ankyrin repeat and SOCS box-containing protein 13 | Asb13 | −1.39 |
| 1456317_at | RIKEN cDNA 1700055N04 gene | 1700055N04Rik | −1.39 |
| 1459301_at | Myeloid ecotropic viral integration site-related gene 1 | Mrg1 | −1.39 |
| 1460459_at | progestin and adipoQ receptor family member V | Paqr5 | −1.39 |
| 1427318_s_at | dysferlin /// fer-1-like 3, myoferlin (C. elegans) | Dysf /// Fer1l3 | −1.40 |
| 1458055_at | Desmocollin 3 | Dsc3 | −1.40 |
| 1418925_at | cadherin EGF LAG seven-pass G-type receptor 1 | Celsr1 | −1.41 |
| 1422662_at | lectin, galactose binding, soluble 8 | Lgals8 | −1.41 |
| 1423034_at | thioredoxin-like 5 | Txnl5 | −1.41 |
| 1426941_at | mucin 15 | Muc15 | −1.41 |
| 1448792_a_at | cytochrome P450, family 2, subfamily f, polypeptide 2 | Cyp2f2 | −1.41 |
| 1425801_x_at | coactosin-like 1 (Dictyostelium) | Cotl1 | −1.42 |
| 1436733_at | RIKEN cDNA E130309F12 gene | E130309F12Rik | −1.42 |
| 1437258_at | Small proline-rich protein 2B | Sprr2a | −1.42 |
| 1448612_at | stratifin | Sfn | −1.42 |
| 1449013_at | eukaryotic elongation factor-2 kinase | Eef2k | −1.42 |
| 1449259_at | RAB3D, member RAS oncogene family | Rab3d | −1.42 |
| 1428083_at | RIKEN cDNA 2310043N10 gene | 2310043N10Rik | −1.43 |
| 1429779_at | eukaryotic translation initiation factor 2C, 4 | Eif2c4 | −1.43 |
| 1435551_at | formin-family protein FHOS2 | FHOS2 | −1.43 |
| 1437927_at | discs, large homolog 2 (Drosophila) | Dlgh2 | −1.43 |
| 1443749_x_at | solute carrier family 1 (glial high affinity glutamate transporter), [symbol] | Slc1a3 | −1.43 |
| 1450699_at | selenium binding protein 1 | Selenbp1 | −1.43 |
| 1418944_at | cysteinyl leukotriene receptor 1 | Cysltr1 | −1.44 |
| 1427165_at | interleukin 13 receptor, alpha 1 | Il13ra1 | −1.44 |
| 1427183_at | epidermal growth factor-containing fibulin-like extracellular matri [symbol] | Efemp1 | −1.44 |
| 1428891_at | RIKEN cDNA 9130213B05 gene | 9130213B05Rik | −1.44 |
| 1437303_at | interleukin 6 signal transducer | Il6st | −1.44 |
| 1451413_at | calpastatin | Cast | −1.44 |
| 1457413_at | Receptor tyrosine kinase-like orphan receptor 1 | Ror1 | −1.44 |
| 1418091_at | transcription factor CP2-like 1 | Tcfcp2l1 | −1.45 |
| 1422603_at | ribonuclease, RNase A family 4 | Rnase4 | −1.45 |
| 1424214_at | RIKEN cDNA 9130213B05 gene | 9130213B05Rik | −1.45 |
| 1427912_at | carbonyl reductase 3 | Cbr3 | −1.45 |
| 1428145_at | acetyl-Coenzyme A acyltransferase 2 (mitochondrial 3-oxoacyl- [symbol] | Acaa2 | −1.45 |
| 1435972_at | calpastatin | Cast | −1.45 |
| 1436092_at | Transcribed sequence with weak similarity to protein pir: S5832 [symbol] | NA | −1.45 |
| 1460732_a_at | periplakin | Ppl | −1.45 |
| 1418762_at | decay accelerating factor 1 | Daf1 | −1.46 |
| 1419086_at | fibroblast growth factor binding protein 1 | Fgfbp1 | −1.46 |
| 1425028_a_at | tropomyosin 2, beta | Tpm2 | −1.46 |
| 1430843_at | glutamate decarboxylase-like 1 | Gadl1 | −1.46 |
| 1434051_s_at | heat shock 70 kDa protein 12A | Hspa12a | −1.46 |
| 1439153_at | IBR domain containing 2 | Ibrdc2 | −1.46 |
| 1436932_at | transcription factor CP2-like 4 | Tfcp2l4 | −1.47 |
| 1448213_at | annexin A1 | Anxa1 | −1.47 |
| 1421492_at | prostaglandin D2 synthase 2, hematopoietic | Ptgds2 | −1.48 |
| 1423771_at | protein kinase C, delta binding protein | Prkcdbp | −1.48 |
| 1433610_at | expressed sequence AA986860 | AA986860 | −1.48 |
| 1416531_at | glutathione S-transferase omega 1 | Gsto1 | −1.49 |
| 1417962_s_at | growth hormone receptor | Ghr | −1.49 |
| 1419027_s_at | glycolipid transfer protein | Gltp | −1.49 |
| 1428146_s_at | acetyl-Coenzyme A acyltransferase 2 (mitochondrial 3-oxoacyl- [symbol] | Acaa2 | −1.49 |
| 1450971_at | growth arrest and DNA-damage-inducible 45 beta | Gadd45b | −1.49 |
| 1452059_at | solute carrier family 35, member F5 | Slc35f5 | −1.49 |
| 1453289_at | eukaryotic translation initiation factor 2C, 4 | Eif2c4 | −1.49 |
| 1457044_at | RIKEN cDNA 4732474O15 gene | 4732474O15Rik | −1.49 |
| 1422906_at | ATP-binding cassette, sub-family G (WHITE), member 2 | Abcg2 | −1.50 |
| 1431248_at | RIKEN cDNA 5031426D15 gene | 5031426D15Rik | −1.50 |
| 1437312_at | bone morphogenetic protein receptor, type 1B | Bmpr1b | −1.50 |
| 1448494_at | growth arrest specific 1 | Gas1 | −1.50 |
| 1449577_x_at | tropomyosin 2, beta | Tpm2 | −1.50 |
| 1451385_at | RIKEN cDNA 2310056P07 gene | 2310056P07Rik | −1.50 |
| 1421456_at | purinergic receptor P2Y, G-protein coupled 1 | P2ry1 | −1.51 |
| 1423606_at | periostin, osteoblast specific factor | Postn | −1.51 |
| 1424239_at | RIKEN cDNA 2310066E14 gene | 2310066E14Rik | −1.51 |
| 1444740_at | Transcribed sequences | NA | −1.51 |
| 1453286_at | plexin A2 | Plxna2 | −1.51 |
| 1419201_at | RIKEN cDNA 2310015I08 gene | 2310015I08Rik | −1.52 |
| 1424612_at | NIPA-like domain containing 2 | Npal2 | −1.52 |
| 1425506_at | myosin, light polypeptide kinase | Mylk | −1.52 |
| 1436365_at | zinc finger and BTB domain containing 36 | Zbtb36 | −1.52 |
| 1436702_at | cDNA sequence BC034068 | BC034068 | −1.52 |
| 1438125_at | RIKEN cDNA C230085N15 gene | C230085N15Rik | −1.52 |
| 1454783_at | interleukin 13 receptor, alpha 1 | Il13ra1 | −1.52 |
| 1440635_at | Transcribed sequences | NA | −1.53 |
| 1455238_at | melanoma associated antigen (mutated) 1-like 1 | Mum1l1 | −1.53 |
| 1426221_at | loss of heterozygosity, 11, chromosomal region 2, gene A homo [symbol] | Loh11cr2a | −1.54 |
| 1429274_at | RIKEN cDNA 2310010M24 gene | 2310010M24Rik | −1.54 |
| 1433719_at | solute carrier family 9 (sodium/hydrogen exchanger), isoform 9 | Slc9a9 | −1.54 |
| 1451739_at | Kruppel-like factor 5 | Klf5 | −1.54 |
| 1435973_at | Transcribed sequences | NA | −1.55 |
| 1450633_at | calmodulin 4 | Calm4 | −1.55 |
| 1452843_at | interleukin 6 signal transducer | Il6st | −1.55 |
| 1441342_at | dipeptidylpeptidase 4 | Dpp4 | −1.56 |
| 1456036_x_at | glutathione S-transferase omega 1 | Gsto1 | −1.56 |
| 1428680_at | CDP-diacylglycerol synthase 1 | Cds1 | −1.57 |
| 1435785_at | EH-domain containing 2 | Ehd2 | −1.57 |
| 1450769_s_at | StAR-related lipid transfer (START) domain containing 5 | Stard5 | −1.57 |
| 1455140_at | expressed sequence AI848332 | AI848332 | −1.57 |
| 1424130_a_at | polymerase I and transcript release factor | Ptrf | −1.59 |
| 1444937_at | NA | NA | −1.59 |
| 1450391_a_at | monoglyceride lipase | Mgll | −1.59 |
| 1450435_at | L1 cell adhesion molecule | L1cam | −1.59 |
| 1421844_at | interleukin 1 receptor accessory protein | Il1rap | −1.60 |
| 1424842_a_at | Rho GTPase activating protein 24 | Arhgap24 | −1.60 |
| 1426245_s_at | microtubule-associated protein, RP/EB family, member 2 | Mapre2 | −1.60 |
| 1426397_at | transforming growth factor, beta receptor II | Tgfbr2 | −1.60 |
| 1442920_at | Kruppel-like factor 3 (basic) | Klf3 | −1.60 |
| 1427011_a_at | LanC (bacterial lantibiotic synthetase component C)-like 1 | Lancl1 | −1.61 |
| 1434957_at | cell adhesion molecule-related/down-regulated by oncogenes | Cdon | −1.61 |
| 1416527_at | RAB32, member RAS oncogene family | Rab32 | −1.62 |
| 1416589_at | secreted acidic cysteine rich glycoprotein | Sparc | −1.62 |
| 1421690_s_at | agouti related protein | Agrp | −1.62 |
| 1431110_at | RIKEN cDNA 5430431D22 gene | 5430431D22Rik | −1.62 |
| 1434866_x_at | carnitine palmitoyltransferase 1a, liver | Cpt1a | −1.62 |
| 1450981_at | calponin 2 | Cnn2 | −1.62 |
| 1416002_x_at | coactosin-like 1 (Dictyostelium) | Cotl1 | −1.63 |
| 1439628_x_at | Rab38, member of RAS oncogene family | Rab38 | −1.63 |
| 1451451_at | grancalcin | Gca | −1.63 |
| 1452614_at | hypothetical gene supported by AF014453 | LOC229672 | −1.63 |
| 1419051_at | OVO homolog-like 1 (Drosophila) | Ovol1 | −1.64 |
| 1422573_at | AMP deaminase 3 | Ampd3 | −1.64 |
| 1427012_at | LanC (bacterial lantibiotic synthetase component C)-like 1 | Lancl1 | −1.64 |
| 1444147_at | proprotein convertase subtilisin/kexin type 2 | Pcsk2 | −1.64 |
| 1456084_x_at | fibromodulin | Fmod | −1.64 |
| 1460406_at | expressed sequence AI427122 | AI427122 | −1.64 |
| 1416007_at | special AT-rich sequence binding protein 1 | Satb1 | −1.65 |
| 1449586_at | plakophilin 1 | Pkp1 | −1.65 |
| 1449848_at | guanine nucleotide binding protein, alpha 14 | Gna14 | −1.65 |
| 1454867_at | meningioma 1 | Mn1 | −1.65 |
| 1428650_at | tensin 1 | Tns1 | −1.66 |
| 1437231_at | SLIT and NTRK-like family, member 6 | Slitrk6 | −1.66 |
| 1438156_x_at | carnitine palmitoyltransferase 1a, liver | Cpt1a | −1.66 |
| 1451989_a_at | microtubule-associated protein, RP/EB family, member 2 | Mapre2 | −1.66 |
| 1455037_at | plexin A2 | Plxna2 | −1.66 |
| 1459529_at | RIKEN cDNA E230016K23 gene | E230016K23Rik | −1.66 |
| 1418318_at | ring finger protein 128 | Rnf128 | −1.67 |
| 1420461_at | macrophage stimulating 1 receptor (c-met-related tyrosine kinas [symbol] | Mst1r | −1.67 |
| 1435693_at | mal, T-cell differentiation protein-like | Mall | −1.67 |
| 1456114_at | CDP-diacylglycerol synthase 1 | Cds1 | −1.67 |
| 1424034_at | RAR-related orphan receptor alpha | Rora | −1.68 |
| 1427164_at | interleukin 13 receptor, alpha 1 | Il13ra1 | −1.68 |
| 1433509_s_at | DNA segment, Chr 6, ERATO Doi 253, expressed | D6Ertd253e | −1.68 |
| 1441214_at | exophilin 5 | Exph5 | −1.68 |
| 1455061_a_at | acetyl-Coenzyme A acyltransferase 2 (mitochondrial 3-oxoacyl- [symbol] | Acaa2 | −1.68 |
| 1417394_at | Kruppel-like factor 4 (gut) | Klf4 | −1.69 |
| 1437829_s_at | eukaryotic elongation factor-2 kinase | Eef2k | −1.69 |
| 1450005_x_at | EGF-like-domain, multiple 9 | Egfl9 | −1.69 |
| 1455627_at | procollagen, type VIII, alpha 1 | Col8a1 | −1.69 |
| 1421970_a_at | glutamate receptor, ionotropic, AMPA2 (alpha 2) | Gria2 | −1.70 |
| 1434146_at | Glutamate receptor, ionotropic, AMPA2 (alpha 2) (Gria2), mRNA | Gria2 | −1.70 |
| 1436236_x_at | coactosin-like 1 (Dictyostelium) | Cotl1 | −1.70 |
| 1427537_at | epiplakin 1 | Eppk1 | −1.71 |
| 1424638_at | cyclin-dependent kinase inhibitor 1A (P21) | Cdkn1a | −1.72 |
| 1439675_at | RIKEN cDNA 4933429D07 gene | 4933429D07Rik | −1.72 |
| 1453345_at | RIKEN cDNA 3830408G10 gene | 3830408G10Rik | −1.72 |
| 1416592_at | glutaredoxin 1 (thioltransferase) | Glrx1 | −1.73 |
| 1436119_at | aldehyde dehydrogenase 1 family, member L2 | Aldh1l2 | −1.73 |
| 1436182_at | special AT-rich sequence binding protein 1 | Satb1 | −1.73 |
| 1420760_s_at | N-myc downstream regulated-like | Ndrl | −1.75 |
| 1434773_a_at | solute carrier family 2 (facilitated glucose transporter), member 1 [symbol] | Slc2a1 | −1.75 |
| 1436590_at | protein phosphatase 1, regulatory (inhibitor) subunit 3B | Ppp1r3b | −1.75 |
| 1445626_at | Lectin, galactose binding, soluble 3 | Lgals3 | −1.75 |
| 1456741_s_at | glycoprotein m6a | Gpm6a | −1.75 |
| 1416200_at | RIKEN cDNA 9230117N10 gene | 9230117N10Rik | −1.76 |
| 1427019_at | protein tyrosine phosphatase, receptor type Z, polypeptide 1 | Ptprz1 | −1.76 |
| 1442434_at | DNA segment, Chr 8, ERATO Doi 82, expressed | D8Ertd82e | −1.76 |
| 1418891_a_at | RAB3D, member RAS oncogene family | Rab3d | −1.77 |
| 1439449_at | special AT-rich sequence binding protein 1 | Satb1 | −1.77 |
| 1455377_at | RIKEN cDNA 4921517B04 gene /// similar to FLJ23033 protein | [symbol] 17B04Rik /// LOC38 | −1.77 |
| 1418086_at | protein phosphatase 1, regulatory (inhibitor) subunit 14A | Ppp1r14a | −1.78 |
| 1424701_at | protocadherin 20 | Pcdh20 | −1.78 |
| 1448392_at | secreted acidic cysteine rich glycoprotein | Sparc | −1.78 |
| 1455288_at | RIKEN cDNA 1110036O03 gene | 1110036O03Rik | −1.78 |
| 1457656_s_at | RIKEN cDNA C230085N15 gene | C230085N15Rik | −1.78 |
| 1426600_at | solute carrier family 2 (facilitated glucose transporter), member 1 [symbol] | Slc2a1 | −1.79 |
| 1443284_at | Special AT-rich sequence binding protein 1 | Satb1 | −1.79 |
| 1422308_a_at | lectin, galactose binding, soluble 7 | Lgals7 | −1.80 |
| 1424698_s_at | grancalcin | Gca | −1.80 |
| 1432976_at | RIKEN cDNA 2310038E17 gene | 2310038E17Rik | −1.80 |
| 1460003_at | expressed sequence AI956758 | AI956758 | −1.81 |
| 1425213_at | RIKEN cDNA 6430514L14 gene | 6430514L14Rik | −1.82 |
| 1458738_at | Guanine nucleotide binding protein, alpha 14 | Gna14 | −1.82 |
| 1439794_at | Netrin 4 (Ntn4), mRNA | Ntn4 | −1.83 |
| 1444392_at | Leprecan-like 1 | Leprel1 | −1.83 |
| 1421679_a_at | cyclin-dependent kinase inhibitor 1A (P21) | Cdkn1a | −1.84 |
| 1429808_at | RIKEN cDNA 1110020C03 gene | 1110020C03Rik | −1.84 |
| 1417700_at | Rab38, member of RAS oncogene family | Rab38 | −1.85 |
| 1420468_at | ankyrin repeat and SOCS box-containing protein 17 | Asb17 | −1.85 |
| 1426599_a_at | solute carrier family 2 (facilitated glucose transporter), member 1 [symbol] | Slc2a1 | −1.85 |
| 1420807_a_at | EGF-like-domain, multiple 9 | Egfl9 | −1.86 |
| 1426243_at | cystathionase (cystathionine gamma-lyase) | Cth | −1.86 |
| 1437813_at | absent in melanoma 1-like | Aim1l | −1.86 |
| 1450782_at | wingless-related MMTV integration site 4 | Wnt4 | −1.86 |
| 1460463_at | RIKEN cDNA 4632413I24 gene | 4632413I24Rik | −1.86 |
| 1420312_s_at | NA | NA | −1.87 |
| 1441687_at | wingless-related MMTV integration site 4 | Wnt4 | −1.87 |
| 1417089_a_at | creatine kinase, mitochondrial 1, ubiquitous | Ckmt1 | −1.88 |
| 1449994_at | epithelial mitogen | Epgn | −1.88 |
| 1431775_at | RIKEN cDNA 3100002H09 gene | 3100002H09Rik | −1.89 |
| 1452284_at | protein tyrosine phosphatase, receptor type Z, polypeptide 1 | Ptprz1 | −1.89 |
| 1455165_at | RAR-related orphan receptor alpha, mRNA (cDNA clone MGC: 5 [symbol] | Rora | −1.89 |
| 1418912_at | plexin domain containing 2 | Plxdc2 | −1.90 |
| 1441917_s_at | transmembrane protein 40 | Tmem40 | −1.90 |
| 1449158_at | potassium channel, subfamily K, member 2 | Kcnk2 | −1.90 |
| 1419204_at | delta-like 1 (Drosophila) | Dll1 | −1.91 |
| 1428622_at | DEP domain containing 6 | Depdc6 | −1.91 |
| 1437019_at | RIKEN cDNA 2200001I15 gene | 2200001I15Rik | −1.91 |
| 1445638_at | Myeloid ecotropic viral integration site-related gene 1 | Mrg1 | −1.91 |
| 1449816_at | sulfotransferase family 5A, member 1 | Sult5a1 | −1.91 |
| 1429067_at | calpain, small subunit 2 | Capns2 | −1.92 |
| 1438570_at | Transcribed sequences | NA | −1.92 |
| 1456174_x_at | N-myc downstream regulated gene 1 | Ndrg1 | −1.92 |
| 1416022_at | fatty acid binding protein 5, epidermal | Fabp5 | −1.93 |
| 1417129_a_at | myeloid ecotropic viral integration site-related gene 1 | Mrg1 | −1.94 |
| 1433203_at | RIKEN cDNA 6030400A10 gene | 6030400A10Rik | −1.94 |
| 1435595_at | RIKEN cDNA 1810011O10 gene | 1810011O10Rik | −1.94 |
| 1455200_at | p21 (CDKN1A)-activated kinase 6 | Pak6 | −1.94 |
| 1417185_at | lymphocyte antigen 6 complex, locus A | Ly6a | −1.95 |
| 1417346_at | PYD and CARD domain containing | Pycard | −1.95 |
| 1430550_at | lipase-like, ab-hydrolase domain containing 3 | Lipl3 | −1.95 |
| 1436325_at | RAR-related orphan receptor alpha | Rora | −1.95 |
| 1448987_at | acetyl-Coenzyme A dehydrogenase, long-chain | Acadl | −1.95 |
| 1455408_at | RIKEN cDNA 4732472I07 gene | 4732472I07Rik | −1.95 |
| 1455794_at | RIKEN cDNA D130058I21 gene | D130058I21Rik | −1.95 |
| 1460038_at | POU domain, class 3, transcription factor 1 | Pou3f1 | −1.95 |
| 1418084_at | neuropilin 1 | Nrp1 | −1.98 |
| 1447791_s_at | guanine nucleotide binding protein, alpha 14 | Gna14 | −1.98 |
| 1440842_at | RIKEN cDNA C230085N15 gene | C230085N15Rik | −1.99 |
| 1442936_at | NA | NA | −1.99 |
| 1451021_a_at | Kruppel-like factor 5 | Klf5 | −2.00 |
| 1416318_at | serine (or cysteine) proteinase inhibitor, clade B, member 1a | Serpinb1a | −2.01 |
| 1440091_at | myeloid ecotropic viral integration site-related gene 1 | Mrg1 | −2.02 |
| 1419905_s_at | hydroxyprostaglandin dehydrogenase 15 (NAD) | Hpgd | −2.05 |
| 1426785_s_at | monoglyceride lipase | Mgll | −2.05 |
| 1434425_at | expressed sequence AI597080 | AI597080 | −2.05 |
| 1436178_at | leprecan-like 1 | Leprel1 | −2.05 |
| 1437699_at | RIKEN cDNA E430014B02 gene | E430014B02Rik | −2.05 |
| 1439819_at | expressed sequence AU015263 | AU015263 | −2.05 |
| 1452031_at | solute carrier family 1 (glial high affinity glutamate transporter), [symbol] | Slc1a3 | −2.05 |
| 1421964_at | Notch gene homolog 3 (Drosophila) | Notch3 | −2.06 |
| 1433768_at | RIKEN cDNA 2410003B16 gene | 2410003B16Rik | −2.06 |
| 1434025_at | Kruppel-like factor 5 | Klf5 | −2.06 |
| 1424852_at | myocyte enhancer factor 2C | Mef2c | −2.07 |
| 1452232_at | UDP-N-acetyl-alpha-D-galactosamine: polypeptide N-acetylgala [symbol] | Galnt7 | −2.07 |
| 1417214_at | RAB27b, member RAS oncogene family | Rab27b | −2.08 |
| 1418365_at | cathepsin H | Ctsh | −2.08 |
| 1424976_at | ras homolog gene family, member V | Rhov | −2.08 |
| 1451308_at | elongation of very long chain fatty acids (FEN1/Elo2, SUR4/Elo3 [symbol] | Elovl4 | −2.08 |
| 1451415_at | RIKEN cDNA 1810011O10 gene | 1810011O10Rik | −2.08 |
| 1447861_x_at | myeloid ecotropic viral integration site-related gene 1 | Mrg1 | −2.09 |
| 1448943_at | neuropilin 1 | Nrp1 | −2.09 |
| 1424208_at | prostaglandin E receptor 4 (subtype EP4) | Ptger4 | −2.11 |
| 1455186_a_at | RIKEN cDNA 1190003J15 gene | 1190003J15Rik | −2.11 |
| 1436838_x_at | coactosin-like 1 (Dictyostelium) | Cotl1 | −2.12 |
| 1415897_a_at | microsomal glutathione S-transferase 1 | Mgst1 | −2.13 |
| 1437811_x_at | Coactosin-like 1 (Dictyostelium) | Cotl1 | −2.13 |
| 1448873_at | occludin | Ocln | −2.14 |
| 1449938_at | placental protein 11 related | Pp11r | −2.16 |
| 1424966_at | transmembrane protein 40 | Tmem40 | −2.17 |
| 1426340_at | solute carrier family 1 (glial high affinity glutamate transporter), n [symbol] | Slc1a3 | −2.17 |
| 1443579_s_at | DEP domain containing 6 | Depdc6 | −2.17 |
| 1454666_at | Kruppel-like factor 3 (basic) | Klf3 | −2.17 |
| 1439915_at | Myeloid ecotropic viral integration site-related gene 1 | Mrg1 | −2.18 |
| 1455573_at | keratin complex 1, acidic, gene 14 | Krt1-14 | −2.18 |
| 1459713_s_at | transmembrane protein 16A | Tmem16a | −2.18 |
| 1419082_at | serine (or cysteine) proteinase inhibitor, clade B, member 2 | Serpinb2 | −2.19 |
| 1437232_at | bactericidal/permeability-increasing protein-like 2 | Bpil2 | −2.20 |
| 1443814_x_at | cathepsin H | Ctsh | −2.20 |
| 1450643_s_at | acyl-CoA synthetase long-chain family member 1 | Acsl1 | −2.20 |
| 1417429_at | flavin containing monooxygenase 1 | Fmo1 | −2.21 |
| 1439492_at | RIKEN cDNA C130053K05 gene | C130053K05Rik | −2.21 |
| 1446764_at | Wingless-related MMTV integration site 3A | Wnt3a | −2.21 |
| 1451348_at | DEP domain containing 6 | Depdc6 | −2.21 |
| 1456815_at | forkhead box N1 | Foxn1 | −2.21 |
| 1426914_at | MARVEL (membrane-associating) domain containing 2 | Mrvldc2 | −2.22 |
| 1429503_at | RIKEN cDNA 2900024C23 gene | 2900024C23Rik | −2.22 |
| 1420385_at | guanine nucleotide binding protein, alpha 14 | Gna14 | −2.23 |
| 1429123_at | RAB27A, member RAS oncogene family | Rab27a | −2.23 |
| 1450744_at | elongation factor RNA polymerase II 2 | EII2 | −2.23 |
| 1418649_at | EGL nine homolog 3 (C. elegans) | Egln3 | −2.24 |
| 1439610_at | RAB27b, member RAS oncogene family | Rab27b | −2.24 |
| 1443138_at | Sulfotransferase family 5A, member 1 | Sult5a1 | −2.24 |
| 1448852_at | regucalcin | Rgn | −2.24 |
| 1417942_at | Ly6/Plaur domain containing 3 | Lypd3 | −2.25 |
| 1431786_s_at | RIKEN cDNA 1190003J15 gene | 1190003J15Rik | −2.25 |
| 1419215_at | aldehyde oxidase 4 | Aox4 | −2.26 |
| 1435076_at | RIKEN cDNA 2310047D13 gene | 2310047D13Rik | −2.26 |
| 1434369_a_at | crystallin, alpha B | Cryab | −2.27 |
| 1419709_at | stefin A3 | Stfa3 | −2.28 |
| 1420944_at | zinc finger protein 185 | Zfp185 | −2.29 |
| 1460302_at | thrombospondin 1 | Thbs1 | −2.29 |
| 1448944_at | neuropilin 1 | Nrp1 | −2.31 |
| 1451716_at | v-maf musculoaponeurotic fibrosarcoma oncogene family, protei [symbol] | Mafb | −2.31 |
| 1452114_s_at | insulin-like growth factor binding protein 5 | Igfbp5 | −2.31 |
| 1423856_at | popeye domain containing 3 | Popdc3 | −2.33 |
| 1434272_at | cytoplasmic polyadenylation element binding protein 2 | Cpeb2 | −2.33 |
| 1430551_s_at | lipase-like, ab-hydrolase domain containing 3 | Lipl3 | −2.34 |
| 1419591_at | melanoma-derived leucine zipper, extra-nuclear factor | Mlze | −2.36 |
| 1421811_at | thrombospondin 1 | Thbs1 | −2.37 |
| 1434411_at | expressed sequence AW743884 | AW743884 | −2.37 |
| 1457632_s_at | myeloid ecotropic viral integration site-related gene 1 | Mrg1 | −2.38 |
| 1425103_at | angiotensin I converting enzyme (peptidyl-dipeptidase A) 2 | Ace2 | −2.41 |
| 1435639_at | RIKEN cDNA 2610528A11 gene | 2610528A11Rik | −2.42 |
| 1451798_at | interleukin 1 receptor antagonist | Il1rn | −2.42 |
| 1439630_x_at | suprabasin | Sbsn | −2.44 |
| 1420364_at | G protein-coupled receptor 87 | Gpr87 | −2.45 |
| 1437463_x_at | transforming growth factor, beta induced | Tgfbi | −2.45 |
| 1437517_x_at | serine (or cysteine) proteinase inhibitor, clade B (ovalbumin), me [symbol] | Serpinb3a | −2.45 |
| 1449064_at | L-threonine dehydrogenase | Tdh | −2.45 |
| 1421604_a_at | Kruppel-like factor 3 (basic) | Klf3 | −2.46 |
| 1434150_a_at | RIKEN cDNA 3300001H21 gene | 3300001H21Rik | −2.46 |
| 1437876_at | Hypothetical gene supported by BC030430 (LOC382098), mRN [symbol] | NA | −2.48 |
| 1449937_at | placental protein 11 related | Pp11r | −2.48 |
| 1451006_at | xanthine dehydrogenase | Xdh | −2.48 |
| 1453239_a_at | ankyrin repeat domain 22 | Ankrd22 | −2.48 |
| 1416697_at | dipeptidylpeptidase 4 | Dpp4 | −2.49 |
| 1422588_at | keratin complex 2, basic, gene 6b | Krt2-6b | −2.49 |
| 1429360_at | Kruppel-like factor 3 (basic) | Klf3 | −2.49 |
| 1442067_at | Receptor tyrosine kinase-like orphan receptor 1 | Ror1 | −2.49 |
| 1448123_s_at | transforming growth factor, beta induced | Tgfbi | −2.49 |
| 1452766_at | RIKEN cDNA 2900041A09 gene | 2900041A09Rik | −2.51 |
| 1415871_at | transforming growth factor, beta induced | Tgfbi | −2.52 |
| 1423883_at | acyl-CoA synthetase long-chain family member 1 | Acsl1 | −2.52 |
| 1425789_s_at | annexin A8 | Anxa8 | −2.52 |
| 1429313_at | receptor tyrosine kinase-like orphan receptor 1 | Ror1 | −2.52 |
| 1424356_a_at | meteorin, glial cell differentiation regulator-like | Metrnl | −2.54 |
| 1443017_at | cytoplasmic polyadenylation element binding protein 2 | Cpeb2 | −2.54 |
| 1446834_at | Cathepsin C | Ctsc | −2.54 |
| 1421965_s_at | Notch gene homolog 3 (Drosophila) | Notch3 | −2.56 |
| 1459897_a_at | suprabasin | Sbsn | −2.56 |
| 1421040_a_at | glutathione S-transferase, alpha 2 (Yc2) | Gsta2 | −2.57 |
| 1421308_at | carbonic anhydrase 13 | Car13 | −2.59 |
| 1460330_at | annexin A3 | Anxa3 | −2.59 |
| 1434295_at | RAS guanyl releasing protein 1 | Rasgrp1 | −2.61 |
| 1454858_x_at | RIKEN cDNA 3300001H21 gene | 3300001H21Rik | −2.62 |
| 1420955_at | visinin-like 1 | Vsnl1 | −2.65 |
| 1427957_at | RIKEN cDNA 9530008L14 gene | 9530008L14Rik | −2.65 |
| 1456250_x_at | transforming growth factor, beta induced | Tgfbi | −2.66 |
| 1429076_a_at | glycerophosphodiester phosphodiesterase domain containing 2 | Gdpd2 | −2.68 |
| 1423414_at | prostaglandin-endoperoxide synthase 1 | Ptgs1 | −2.69 |
| 1416382_at | cathepsin C | Ctsc | −2.70 |
| 1441094_at | alcohol dehydrogenase 6B (class V) | Adh6b | −2.70 |
| 1452166_a_at | keratin complex 1, acidic, gene 10 | Krt1-10 | −2.73 |
| 1426208_x_at | pleiomorphic adenoma gene-like 1 | Plagl1 | −2.74 |
| 1443926_at | Myeloid ecotropic viral integration site-related gene 1 | Mrg1 | −2.74 |
| 1454159_a_at | insulin-like growth factor binding protein 2 | Igfbp2 | −2.74 |
| 1423323_at | tumor-associated calcium signal transducer 2 | Tacstd2 | −2.76 |
| 1417852_x_at | chloride channel calcium activated 1 | Clca1 | −2.79 |
| 1459973_x_at | dipeptidylpeptidase 4 | Dpp4 | −2.80 |
| 1460591_at | estrogen receptor 1 (alpha) | Esr1 | −2.80 |
| 1438109_at | chloride channel calcium activated 5 | Clca5 | −2.81 |
| 1447891_at | NA | NA | −2.81 |
| 1434151_at | RIKEN cDNA 3300001H21 gene | 3300001H21Rik | −2.82 |
| 1417702_a_at | histamine N-methyltransferase | Hnmt | −2.83 |
| 1419492_s_at | defensin beta 1 | Defb1 | −2.83 |
| 1421307_at | carbonic anhydrase 13 | Car13 | −2.83 |
| 1435761_at | stefin A3 | Stfa3 | −2.83 |
| 1425102_a_at | angiotensin I converting enzyme (peptidyl-dipeptidase A) 2 | Ace2 | −2.87 |
| 1439528_at | RIKEN cDNA 4833423E24 gene | 4833423E24Rik | −2.88 |
| 1426708_at | anthrax toxin receptor 2 | Antxr2 | −2.92 |
| 1435436_at | Transcribed sequences | NA | −2.92 |
| 1416225_at | alcohol dehydrogenase 1 (class I) | Adh1 | −2.93 |
| 1450460_at | aquaporin 3 | Aqp3 | −2.95 |
| 1449500_at | serine (or cysteine) proteinase inhibitor, clade B, member 7 | Serpinb7 | −2.96 |
| 1424968_at | RIKEN cDNA 2210023G05 gene | 2210023G05Rik | −2.99 |
| 1431211_s_at | thioesterase superfamily member 5 | Them5 | −2.99 |
| 1456539_at | Transcribed sequence with strong similarity to protein sp: P0072 [symbol] | NA | −3.02 |
| 1422587_at | transmembrane protein 45a | Tmem45a | −3.09 |
| 1419491_at | defensin beta 1 | Defb1 | −3.12 |
| 1438474_at | ankyrin repeat domain 35 | Ankrd35 | −3.12 |
| 1434227_at | keratinocyte differentiation associated protein | Krtdap | −3.16 |
| 1436520_at | expressed sequence AI450948 | AI450948 | −3.18 |
| 1441909_s_at | RIKEN cDNA 9530066K23 gene | 9530066K23Rik | −3.20 |
| 1417853_at | chloride channel calcium activated 1 | Clca1 | −3.26 |
| 1459898_at | suprabasin | Sbsn | −3.31 |
| 1441341_at | Transcribed sequences | NA | −3.32 |
| 1437939_s_at | Cathepsin C | Ctsc | −3.34 |
| 1424713_at | calmodulin-like 4 | Calml4 | −3.36 |
| 1422008_a_at | aquaporin 3 | Aqp3 | −3.38 |
| 1435760_at | cystatin A | MGI: 3524930 | −3.43 |
| 1448932_at | keratin complex 1, acidic, gene 16 | Krt1-16 | −3.44 |
| 1417732_at | annexin A8 | Anxa8 | −3.45 |
| 1457025_at | RIKEN cDNA 4833413O15 gene | 4833413O15Rik | −3.46 |
| 1440523_at | retinal short chain dehydrogenase reductase | Rdhe2 | −3.53 |
| 1441384_at | Transcribed sequences | NA | −3.55 |
| 1417408_at | coagulation factor III | F3 | −3.63 |
| 1448152_at | insulin-like growth factor 2 | Igf2 | −3.64 |
| 1458000_at | desmoglein 1 alpha | Dsg1a | −3.70 |
| 1426808_at | lectin, galactose binding, soluble 3 | Lgals3 | −3.74 |
| 1416930_at | lymphocyte antigen 6 complex, locus D | Ly6d | −3.80 |
| 1419463_at | chloride channel calcium activated 2 | Clca2 | −3.86 |
| 1455519_at | desmoglein 1 alpha | Dsg1a | −3.90 |
| 1460259_s_at | chloride channel calcium activated 1 | Clca1 | −3.92 |
| 1439620_at | carbonic anhydrase 13 | Car13 | −3.93 |
| 1422481_at | keratin complex 2, basic, gene 1 | Krt2-1 | −4.06 |
| 1456203_at | RIKEN cDNA 1110020A10 gene | 1110020A10Rik | −4.35 |
| 1424306_at | elongation of very long chain fatty acids (FEN1/Elo2, SUR4/Elo3 [symbol] | Elovl4 | −4.40 |
| 1439183_at | N-acylsphingosine amidohydrolase (alkaline ceramidase) 3 | Asah3 | −4.57 |
| 1424265_at | N-acetylneuraminate pyruvate lyase | Npl | −4.60 |
| 1453801_at | thioesterase superfamily member 5 | Them5 | −4.68 |
| 1437578_at | chloride channel calcium activated 2 | Clca2 | −4.86 |
| 1429297_at | serine (or cysteine) proteinase inhibitor, clade B (ovalbumin), me [symbol] | Serpinb12 | −4.97 |
| 1422939_at | serine (or cysteine) proteinase inhibitor, clade B (ovalbumin), me [symbol] | Serpinb3b | −5.05 |
| 1422940_x_at | serine (or cysteine) proteinase inhibitor, clade B (ovalbumin), me [symbol] | Serpinb3b | −5.07 |
| 1436448_a_at | prostaglandin-endoperoxide synthase 1 | Ptgs1 | −5.11 |

[symbol]

indicates data missing or illegible when filed

TABLE 2
PCAD+

Hair Follicle
Role
Morphogens/Signaling
Bmp2
Bmp4*
Dkk3^{#}
Dkk4
Edar
Fgfr1^{#}
Fzd7^{#}
Hhip
Inhbb^{#}
Ptch2
Pdgfa
Pdgfr1
Sgk
Shh
Tgfa
Tgfb2
Tgfb3
Wnt5a
Wif1^{#}
Transcription
Factors
Bnc1^{#}
Bnc2
Cutl1*
Gli1
Hoxc13
Nmyc1
Nfatc1^{#}
Sox9^{#}
Trps1^{#}
Vdr^{#}
Bcl6*
Bach2^{#}
Dach1
Dlx2
Egr2*
Egr3
Foxc1^{#}
Gtf2ird1^{#}
Hmga2^{#}
Id2^{#}
Id4^{#}
Isl1
Lhx2^{#}
Lbh
Lass4
Mitf^{#}
Myb*
Nfe2l3*
Nr4a1
Nap1l5
Prdm1
Prdm5
Rai14^{#}
Runx1^{#}
Runx2*
Scmh1^{#}
Six4
Sox13^{#}
Sox2
Sox21*
Tbx1^{#}
Sp5
Tcf7
Tcfl5
Zfp566

PCAD−

Epidermal Role
Adhesion/Structural
Cldn1*
Dsc2^{#}
Dsg1a
Evpl
Eppk1*
Jup
Krt1-10*
Krt1-14*
Krt1-15
Krt1-16*
Krt2-1*
Krt2-5
Krt2-6a^{#}
Krt2-6b
Ocln
Ppl
pkp1*
Differentiation
BmpR1b
Dll1*
Dtx2
Jag1^{#}
Krtdap*
Notch3*
Sprr2a
Sfn
Sbsn
Transcription
Factors
Esr1
Foxn1
Grhl1*
Grhl3
Klf4
Klf5*
Myc^{#}
Ovol1*
Pou3f1*
Stat1
Tcfap2e
Ascl2*
Aff1
Ankrd22*
Ghr*
Ell2^{#}
Hoxa1*
Mllt2h^{#}
Irx3
Klf12
Klf3
Mafb*
Msl2
Mrg1*
Mef2c
Nfia
Nrip3^{#}
Nr2f2
Ror1
Rora*
Satb1*
Spib
Sox6*
Sox7*
Tcfcp2l1
Note:
Expression of K14 and K5 genes was previously shown to be downregulated as basal embryonic epidermal cells commit to make a hair follicle; expression of K1 and K10 are typically expressed in terminally differentiating epidermal cells but a few basal cells have been shown to induce expression of these genes prior to detachment from the basement membrane. Thus, the reduced expressions of K14/K5 and K10/K1 in PCAD+ placodes relative to PCAD− basal cells (or in bulge cells relative to all skin epithelial cells) are in agreement with these points.

What is claimed is:

1. A method for modulating hair growth comprising regulating the expression or activity of Lhx2 so that hair growth is stimulated or inhibited.

2. A method for identifying an agent which modulates hair growth comprising contacting a test cell expressing a reporter operably linked to an Lhx2 promoter with an agent and detecting expression of the reporter in the test cell, wherein a decrease in reporter expression is indicative of an agent which stimulates hair growth and an increase in reporter expression is indicative of an agent which inhibits hair growth.
